(19) United States
(12) Patent Application Publication
(10) Pub. No.: US 2003/0139332 A1
(43) Pub. Date: Jul. 24, 2003

(54) Use of matrix metalloproteinase inhibitors to mitigate nerve damage

(75) Inventors: Linda Jeanne Noble, San Francisco, CA (US); Frances Muriel Donovan, San Francisco, CA (US); Zena Werb, San Francisco, CA (US)

Correspondence Address: QUINE INTELLECTUAL PROPERTY LAW GROUP, P.C., P O BOX 458, ALAMEDA, CA, 94501, US

(73) Assignee: The Regents of the University of California

(21) Appl. No.: 10/192,397

(22) Filed: Jul. 9, 2002

Related U.S. Application Data
(60) Provisional application No. 60/304,306, filed on Jul. 9, 2001.

(51) Int. Cl. A61K 39/395; A61K 31/192; A61K 31/19; A61K 31/18; A61K 31/16

(52) U.S. Cl. 514/12; 514/570; 514/602; 514/616; 514/575; 424/146.1

(57) ABSTRACT

This invention pertains to the discovery that inhibitors of matrix metalloproteinases (e.g. MMP-9) can reduce neurological damage (e.g. secondary damage) following trauma to nervous tissue in a mammal, and/or reduce abnormal vascular permeability associated with spinal cord injury, and/or improving recovery of neurological function following injury to neurological tissue. Methods of use of matrix metalloproteinase inhibitors for such applications are provided.

### CROSS-REFERENCE TO RELATED APPLICATIONS

This application claims priority to and benefit of U.S. Ser. No. 60/304,306, filed on Jul. 9, 2001, which is incorporated herein by reference in its entirety for all purposes.

### STATEMENT AS TO RIGHTS TO INVENTIONS MADE UNDER FEDERALLY SPONSORED RESEARCH AND DEVELOPMENT

This work was supported in part by Grant Nos NS39278, T32 ES07106, and NS 39847. The Government of the United States of America may have certain rights in this invention.

### FIELD OF THE INVENTION

This invention is in the fields of neurology and pharmacology, and relates to drugs that can minimize brain injury to the central nervous system (e.g. brain, spinal cord, etc.) due to various causes, such as traumatic head injury or crises such as stroke, cardiac arrest, or asphyxiation.

### BACKGROUND OF THE INVENTION

Each year there are approximately 10,000 spinal cord injuries that result in permanent disabilities. Motor vehicle accidents are the leading cause of spinal cord injury, followed by acts of violence, falls, and sports. The majority of injuries occur at the mid-cervical and upper thoracic regions of the spinal cord and lead to extremely debilitating conditions.

Approximately 45% of the injuries result in total or complete loss of sensation and function below the level of injury. The remaining 55% of the injuries result in partial loss of sensation and function below the level of injury. Such injuries are incomplete. In incomplete spinal cord injuries, the primary traumatic injury can destroy as much as 90% of the axons in the spinal cord. However, such patients can still recover substantial function as a result of the axons that are spared at the injury site.

The primary trauma to the spinal cord causes a central hemorrhagic necrosis. The central part of the spinal cord, i.e., the gray matter dies first. Generally a rim of white matter containing myelinated axons is preserved. Following the initial injury, a series of degenerative processes which promote tissue damage beyond the original site of injury are initiated. This is referred to as secondary injury.

There is considerable evidence that functional recovery after spinal cord injury is not simply the consequences of the initial mechanical destruction of tissue but is also attributed to the evolution of complex secondary events that contribute to early as well as delayed cell injury.

At present, the prevalent method used to reduce or minimize the damage resulting from this secondary injury is intravenous injection of the glucocorticoid methylprednisolone. Methylprednisolone is a potent free radical scavenger which may also serve to reduce inflammation of the central nervous system. Methylprednisolone is administered to the patient in high doses (e.g., ˜30 mg/kg body weight) shortly after injury, typically within the first 8 hours. Unfortunately, prolonged administration of glucocorticoids has adverse systemic effects (e.g. increased incidence of sepsis and pneumonia) and is limited in its therapeutic window.

### SUMMARY OF THE INVENTION

This invention pertains to the discovery that inhibitors of expression and/or activity of matrix metalloproteinases (e.g. MMP-9) can reduce neurological damage following trauma to nervous tissue in a mammal, and/or reduce abnormal vascular permeability associated with spinal cord injury, and/or improve recovery of neurological function following injury to neurological tissue. Methods of use of matrix metalloproteinase inhibitors for such applications are provided.

Thus, in one embodiment, this invention provides a method of reducing neurological damage following trauma to nervous tissue in a mammal. The method involves inhibiting activity or expression of a matrix metalloproteinase (e.g. MMP-9) in said mammal before, during, or after said trauma. The method can comprise administering to the mammal (e.g. a human, a non-human mammal) a matrix metalloproteinase inhibitor (MMPI) during or after the trauma. In certain embodiments, the mammal is a human afflicted with or following a stroke and/or a human afflicted with a spinal cord injury.

In certain embodiments, the matrix metalloproteinase inhibitor(s) (MMPI) can include, but are not limited to, one or more of the following: a 4-biarylbutyric acid derivative, a 5-biarylpentanoic acid derivative, Fenbufen, peptide MMPIs, a hydroxamic acid, a tricyclic butyric acid derivative, a biphenyl butyric acid derivative, a heterocyclic substituted phenyl butyric acid derivative, a sulfonamide derivative, a succinamide MMP inhibitor, a sulfonated amino acid derivatives, and a neutralizing anti-MMP antibody. In certain embodiments, teh matrix metalloproteinase inhibitor (MMPI) includes one or more of the following: BAY12-9566, batimastat (BB-94), TIMP-1, prinomastat (AG-3340), and RO 31-9790. In certain embodiments, the MMPI(s) are provided in unit dosage form(s) at a concentration sufficient to inhibit neurological damage following trauma to nervous tissue in the mammal. Various neurological traumas include, but are not limited to ischemia, spinal cord injury, cranial injury, physical trauma to the head or spinal cord; a brain concussion; ischemic stroke caused by thrombosis or embolism; cerebral hemorrhage, general circulatory failure, circulatory disruption caused by cardiac arrest, hemodynamic shock caused by loss of blood due to injury or hemorrhage elsewhere in the body; vasculatory damage caused by vascular disease, bacterial infection, viral infection, fungal infection, cerebral or spinal tumors, glial cell swelling, hypoxic injury to the brain caused by respiratory disruption, and post-operative brain injury or stress.

In certain embodiments, the trauma comprises a spinal cord injury. In certain embodiments, the trauma comprises a brain injury. In certain embodiments, the trauma comprises a motor nerve injury and/or a sensory nerve injury.

In certain embodiments, the MMPI is a specific or a non-specific inhibitor of MMP-9. The MMPI can be an MMPI that is not an inhibitor of MMP-2 activity. In certain embodiments, the MMPI is not an inhibitor of the activity of any matrix metalloproteinase other than MMP-9.

In certain embodiments, the administering is for up to 10 days, preferably for up to 5 days following the trauma.

The method can comprise administering to the mammal an agent that inhibits expression of a matrix metalloproteinase. In certain embodiments, the agent is not a glucocorticoid. In certain embodiments, the agent is not methylprednisolone.

In another embodiment, this invention provides a method of reducing abnormal vascular permeability associated with brain or spinal cord injury. The method involves comprising administering to a mammal in need thereof an inhibitor of matrix metalloporteinsase expression or activity in an amount sufficient to reduce abnormal vascular permeability associate with or following the spinal cord injury. In certain embodiments, the method comprises administering one or more matrix metalloproteinase inhibitor(s) (MMPI(s)) (e.g., 4-biarylbutyric acid derivative, a 5-biarylpentanoic acid derivative, Fenbufen, peptide MMPIs, a hydroxamic acid, a tricyclic butyric acid derivative, a biphenyl butyric acid derivative, a heterocyclic substituted phenyl butyric acid derivative, a sulfonamide derivative, a succinamide MMP inhibitor, a sulfonated amino acid derivatives, a neutralizing anti-MMP antibody, etc.). In certain embodiments the MMPI comprises an MMPI selected from the group consisting of BAY12-9566, batimastat (BB-94), TIMP-1, prinomastat (AG-3340), and RO 31-9790. The MMPI can be provided in a unit dosage form, e.g., at a concentration sufficient to inhibit abnormal vascular permeability following spinal cord injury.

In certain embodiments, the MMPI is a specific or a non-specific inhibitor of MMP-9. The MMPI can be an MMPI that is not an inhibitor of MMP-2 activity. In certain embodiments, the MMPI is not an inhibitor of the activity of any matrix metalloproteinase other than MMP-9.

The mammal can be a human or a non-human mammal. In certain embodiments, the mammal is a human afflicted with or following a stroke and/or a human afflicted with a spinal cord injury and/or a human afflicted with a brain injury.

This invention also provides a method of improving recovery of neurological function following injury to neurological tissue. The method involves comprising administering to a mammal in need thereof an inhibitor of a matrix metalloproteinase expression or activity in an amount sufficient to improve recovery of neurological function following said injury. In certain embodiments, the injury comprises a spinal cord injury. In certain embodiments, the injury comprises a brain injury. In certain embodiments, the injury comprises a motor nerve injury. In certain embodiments, the injury comprises a sensory nerve injury. The recovery can comprise recovery of locomotor function and/or sensory function. Suitable metalloproteinase inhibitors (MMPI) include, but are not limited to one or more of the MMPIs described herein. In certain embodiments, the MMPI(s) are provided in a unit dosage form at a concentration sufficient to promote recovery of locomotor function following spinal cord injury.

In certain embodiments, the injury comprises an injury associated with a condition selected from the group consisting of ischemia, spinal cord injury, cranial injury, physical trauma to the head or spinal cord; a brain concussion; ischemic stroke caused by thrombosis or embolism; cerebral hemorrhage, general circulatory failure, circulatory disruption caused by cardiac arrest, hemodynamic shock caused by loss of blood due to injury or hemorrhage elsewhere in the body; vasculatory damage caused by vascular disease, bacterial infection, viral infection, fungal infection, cerebral or spinal tumors, glial cell swelling, hypoxic injury to the brain caused by respiratory disruption, and post-operative brain injury or stress.

The mammal can be a human or a non-human mammal. In certain embodiments, the mammal is a human afflicted with or following a stroke and/or a human afflicted with a spinal cord injury and/or a human afflicted with a brain injury.

In any of the methods described herein, the method can comprise administering to the mammal an inhibitor of MMP (e.g. MMP-9) expression or activity and a prophylactically or therapeutically effective amount of one or more anti-inflammatory agents during or after the trauma. In certain embodiments, the anti-inflammatory agent is a non-steroidal anti-inflammatory drug (e.g. aspirin, ibuprofen, diclofenac, nabumetone, naproxen, or ketoproten). In various embodiments, the method can comprise administering to the mammal an inhibitor of MMP (e.g. MMP-9) expression or activity and a prophylactically or therapeutically effective amount of one or more anti-convulsive agents (e.g., carbamazepine (Tegretol®), phenobarbital, primidone, phenytoin (Dilantin®), valproic acid (Depakote®), ethosuximide, clonazepam, levitracetam, gabapentin, gabatril, lamotrigine, oxcarbazepine or topiramate, etc.) during or after the trauma.

In any of the embodiments the mammal can be a human or non-human mammal that is not presently and/or that has never been diagnosed as having cancer and/or metastatic disease.

This invention also provides a kit for reducing neurological damage following trauma to nervous tissue in a mammal. The kit can include an inhibitor of matrix metalloproteinase expression or activity (e.g. an MMPI) and instructional materials teaching the use of the inhibitor for reducing neurological damage following trauma to nervous tissue in a mammal. In certain embodiments, the kit comprises one or more matrix metalloproteinase inhibitor(s) (MMPI(s)) is selected from the group consisting of a 4-biarylbutyric acid derivative, a 5-biarylpentanoic acid derivative, Fenbufen, peptide MMPIs, a hydroxamic acid, a tricyclic butyric acid derivative, a biphenyl butyric acid derivative, a heterocyclic substituted phenyl butyric acid derivative, a sulfonamide derivative, a succinamide MMP inhibitor, a sulfonated amino acid derivatives, and a neutralizing anti-MMP antibody. In certain embodiments, the matrix metalloproteinase inhibitor (MMPI) is selected from the group consisting of BAY12-9566, batimastat (BB-94), TIMP-1, prinomastat (AG-3340), and RO 31-9790. The MMPI(s) can be provided in a unit dosage form at a concentration sufficient to inhibit secondary neurological damage following said trauma. In various embodiments, the trauma is a trauma associated with ischemia, spinal cord injury, cranial injury, physical trauma to the head or spinal cord; a brain concussion; ischemic stroke caused by thrombosis or embolism; cerebral hemorrhage, general circulatory failure, circulatory disruption caused by cardiac arrest, hemodynamic shock caused by loss of blood due to injury or hemorrhage elsewhere in the body; vasculatory damage caused by vascular disease, bacterial infection, viral infection, fungal infection, cerebral or spinal tumors, glial cell swelling, hypoxic injury to the brain caused by respiratory disruption, and/or post-operative brain injury or stress. In certain embodiments, the trauma is a spinal cord injury. The inhibitor can be a specific inhibitor of MMP-9. The mammal can be a human (e.g. a human afflicted with or following a stroke and/or a human afflicted with a spinal cord injury)or a non-human mammal.

In still another embodiment, this invention provides, in a mammal diagnosed as having or as at risk from secondary neurological damage, an exogenously applied inhibitor of a matrix metalloproteinase activity or expression. The mammal is preferably a mammal not diagnosed as having a cancer. In certain embodiments, the inhibitor is an inhibitor of MMP-9 expression or activity.

### DEFINITIONS

Matrix metalloproteinases are well known to those of skill in the art. Matrix metalloproteinases (MMPs) are a group of enzymes that have been implicated in the pathological destruction of connective tissue and basement membranes (see, e.g., Woessner (1991) FASEB J. 5: 2145; Birkedal-Hansen et al. (1993) Crit. Rev. Oral Biol. Med. 4: 197; Cawston (1996) Pharmacol. Ther. 70: 163; Powell and Matrisian (1996) Cur. Top. Microbiol. and Immunol. 213: 1). These zinc containing endopeptidases consist of several subsets of enzymes including collagenases, stromelysins and gelatinases, and include, but are not limited to MMP-1, MMP-2, MMP-3, MMP-8, MMP-9, MMP-12, MMP-13. IN particular, MMP-1 refers to interstitial collagenase; MMP-2 refers to Gelatinase A; MMP-3 refers to stromelysin; MMP-7 refers to matrilysin; and MMP-9 refers to Gelatinase B. Various references discussing MMPs include, but are not limited to Noha et al. (2000) J. Neuro-Oncology, 48(3): 217-223; Asahi et al. (2000) J. Cerebral Blood Flow and Metabolism, 20(12):1681-1689; Lou et al. (1999) Laboratory Investigation, 79(8): 1015-1025; Romanic et al. (1998) Stroke, 29(5): 1020-1030; and Rosenberg and Navratil (1997) Neurology, 48(4): 921-926.

The terms “matrix metalloproteinase 9” or “MMP-9” are used to refere to the MMP also known as Gelatinase B. MMP-9 degrades gelatin (denatured collagens), collagen IV, V, XI, elastin, vitronectin, myelin basic protein and other substrates (Vu and Werb (1998) Gelatinase B: structure, regulation, and function. San Diego: Academic Press). This protease is predominantly expressed by inflammatory cells including macrophages, lymphocytes, and neutrophils as well as endothelial cells (Mainardi et al. (1984) Collagen and Related Research 4:479-492; Hibbs et al. (1987) J. Clin. Invest., 80:1644-1650; Murphy et al. (1989) Biolchem. J., 258:463-472; Wilhelm et al. (1989) [published erratum appears in J Biol Chem 1990 December; 265(36):22570]. J. Biol, Chem., 264:17213-17221). Recent studies suggest that MMP-9 inactivates alpha 1-antitrypsin, the primary physiologic inhibitor of leukocyte elastase, a step that is central to leukocyte migration (Liu et al. (1998) J. Exp. Med., 188:475-482). In the central nervous system, there is a low constitutive expression of MMP-9 in microglia, astrocytes and hippocampal neurons, and it can be induced in astrocytes, microglia/macrophages and hippocampal cells (Backstrom et al. (1996) J. Neurosci., 16:7910-7919; Cuzner et al. (1996) J. Neuropathology Exp. Neurol., 55:1194-1204; Gottschall and Deb (1996) Neuroimmunomodulation 3:69-75; Liu et al. (1998) J. Exp. Med., 188:475-482; Yong et al. (2001) Nat Rev Neurosci 2:502-511).

A “matrix metalloproteinase inhibitor” as used herein is any chemical compound that inhibits (reduces or eliminates) the hydrolytic activity of at least one matrix metalloproteinase enzyme that is naturally occurring in a mammal. Such compounds are also referred to as “MMP inhibitors” or “MMPIs”. Preferred matrix metalloproteinase inhibitors inhibit the hydrolytic activity of at least one matrix metalloproteinase enzyme by at least five percent, preferably by at least 10 percent, more preferably by at least 20 percent and most preferably by at least 30 percent, at least 40 percent or at least 50 percent the hydrolytic activity of at least one matrix metalloproteinase enzyme in a mammal. Numerous matrix metalloproteinase inhibitors are known, and it is believed that all are useful in the method of this invention. For example, 4-biarylbutyric and 5-biarylpentanoic acid derivatives are described in WO 96/15096. In one embodiment of the invention, an MMP-9 specific inhibitor is used. In specific embodiments, an MMP-9 specific inhibitor inhibits the the hydrolytic activity of MMP-9 by at least one times, two times, five times, 10 times, 20 times, 50 times, 100 times, or 500 times more than it inhibits other MMPs. In another embodiment, the inhibitor inhibits MMP-9, but does not inhibit MMP-2. In specific embodiments, the inhibitor inhibits the hydrolytic activity of MMP-9 by at least one times, two times, five times, 10 times, 20 times, 50 times, 100 times, or 500 times more than it inhibits MMP-2.

The term “mammal” is intended to include humans as well as non-human mammals. Thus the methods of this invention are intended to include veterinary applications as well as human medical applications. Preferred non-human mammals include, but are not limited to equines, felines, canines, porcines, and the like.

The phrase “inhibit neurological damage” refers to a reduction or elimination of neurological damage. In certain embodiments the phrase refers to secondary neurological damage, e.g. damage that occurs subsequent to a primary injury or trauma. The inhibition refers to a partial or complete reduction in damage (e.g. as measured by any convenient index, e.g. lesion area, degree of locomotor recovery, etc.). In preferred embodiments, the reduction is a statistically significant reduction (e.g. at the 90%, 95%, or 99% confidence level), e.g. as determined using any statistical test suited for the data set provided (e.g. t-test, analysis of variance (ANOVA), semiparametric techniques, non-parametric techniques (e.g. Wilcoxon Mann-Whitney Test, Wilcoxon Signed Ranks Test, Sign Test, Kruskal-Wallis Test, etc.). Preferably the statistically significant change is significant at least at the 85%, more preferably at least at the 90%, still more preferably at least at the 95%, and most preferably at least at the 98% or 99% confidence level). In certain embodiments, the change is at least a 10% change, preferably at least a 20% change, more preferably at least a 50% change and most preferably at least a 90% change. Preferred reductions include at least a 5% reduction, more preferably at least a 10% reduction, and most preferably at least a 15% or 20% reduction in neurological damage.

As used herein, an “antibody” refers to a protein or glycoprotein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies exist as intact immunoglobulins or as a number of well characterized fragments produced by digestion with various peptidases. Thus, for example, pepsin digests an antibody below (i.e. toward the Fc domain) the disulfide linkages in the hinge region to produce F(ab)′2, a dimer of Fab which itself is a light chain joined to V_{H}-C_{H}1 by a disulfide bond. The F(ab)′2 may be reduced under mild conditions to break the disulfide linkage in the hinge region thereby converting the (Fab′)2 dimer into an Fab′ monomer. The Fab′ monomer is essentially a Fab with part of the hinge region (see, Paul (1993) Fundamental Immunology, Raven Press, N.Y. for a more detailed description of other antibody fragments). While various antibody fragments are defined in terms of the digestion of an intact antibody, one of skill will appreciate that such fragments may be synthesized de novo either chemically, by utilizing recombinant DNA methodology, or by “phage display” methods (see, e.g., Vaughan et al. (1996) Nature Biotechnology, 14(3): 309-314, and PCT/US96/10287). Preferred antibodies include single chain antibodies, e.g., single chain Fv (scFv) antibodies in which a variable heavy and a variable light chain are joined together (directly or through a peptide linker) to form a continuous polypeptide.

An anti-MMP antibody is an antibody that specifically binds an MMP (e.g. MMP-9). Preferred anti-MMP antibodies inhibit activity of the MMP(s) to which they bind.

### BRIEF DESCRIPTION OF THE DRAWINGS

FIGS. 1A and 1B show that the time course of MMP-9 activity increases after spinal cord injury. A 3-mm length of spinal cord, centered over the impact site, was flash frozen and homogenized. Soluble fractions were analyzed by (FIG. 1A) gelatin zymography or (FIG. 1B) in reverse gelatin zymography. (FIG. 1A) MMP-9 activity increases acutely after spinal cord injury and decreases by 1 week post injury. Note that the absence of MMP-9 activity in the null mouse does not result in a compensatory increase in MMP-2 activity in the injured spinal cord. 1,10 phenanthroline, a general inhibitor of metalloproteinases, completely blocks the inactive and active forms, thus confirming the specificity of these molecules. The position of migration of active and zymogen forms of MMP-9, and MMP-2 and the MMP-9/TIMP-1 complexes determined from standards are marked. (FIG. 1B) TIMP activity, seen by reverse zymography, is unchanged after spinal cord injury. The position of migration of TIMP-1 and -2 determined from standards are marked, as well as the migration of proMMP-9 and active MMP-9.

FIG. 2, panels A-F illustrate immunolocalization of MMP-9. Immunolocalization of MM_-9 is shown in the uninjured spinal cord and at 24 hours post injury. (panels A, B) uninjured spinal cord; (panels C-F) injured spinal cord. (panels A-D), anti-MMP-9 in the wildtype mouse; (panels E,F) anti-MMP-9 in the MMP-9 null mouse. MMP-9, visualized by HRP immunohistochemistry, is localized in meninges (arrowhead, A) and ventral horn motoneurons (panel B, arrowheads). After spinal cord injury there is prominent expression of MMP-9 at the lesioned epicenter (panel C). At higher magnification MMP-9 is localized within vascular structures (D, arrows) as well as in round cells, bearing no processes (panel D, arrowheads). There is no staining within the epicenter (E) or motoneurons in the adjacent penumbral zone (panel F, arrowhead) in the MMP-9 null animal. Panels A, C, E. Scale Bar=500 μm. Panels B, D. Scale Bar=50 μm. Panel F Scale Bar=100 μm

FIG. 3, panels A-F show immunolocalization of MMP-9 at 24-72 hours post injury. Based upon double immunofluorescence, MMP-9 (panels A, B, E) is localized in blood vessels (panel C, PECAM immunolocalization) macrophages (panel D, F4/80 immunolocalization), and astrocytes (panel F, glial fibrillary acidic protein immunolocalization). Controls had no immunofluorescence (not shown) panels A-D. Scale Bar=50 μm. Panels panels E, F. Scale Bar=100 μm

FIG. 4, panels A-F shows localization of gelatinolytic activity in situ after spinal cord injury. Unfixed spinal cords from the mice (uninjured or at 24 hours post injury), frozen, and cryosections prepared for in situ gelatin zymography as described herein. Fluorescence is indicative of gelatinolytic activity. In the uninjured wildtype spinal cord, small amounts of gelatinase activity are identified in the meninges (arrowheads, panel A). After spinal cord injury gelatinase activity is prominent in the meninges (arrowhead, panel B) as well as within the epicenter (panel C). The gelatinase activity within the epicenter is localized at least in part to blood vessels (panel D). In the MMP-9 null, injured mouse (panels E, F), gelatinase activity is not as reobuts in the epicenter (panel E). Activity still appears in the meninges (panels E and F, arrowheads) and blood vessels (panel F, arrow). Panels A, B. Scale Bar=500 μm. Panel C Scale Bar=100 μm. Panel D Scale Bar=50 μm.

FIG. 5, panels A-D show blood-spinal cord barrier disruption to horseradish peroxidase (HRP) at 24 hours post injury in the wild type (panels A, C) and the MMP-9 null mice (panels B, D). The lateral white matter is characterized by radial spokes of intraparenchymal hemorrhage (arrows, A, B). HRP, appearing as a dark brown diffuse reaction product, is more pronounced in the wild type (panel C) as compared to the MMP-9 null (panel D) spinal cord. A, B. Scale Bar=500 μm C, D. Scale Bar=100 μm

FIG. 6 shows the pattern of blood-spinal cord barrier disruption to horseradish peroxidase (HRP) at 24 hours post injury in MMP-9 wild type and null mice and in mice treated with GM6001. The relative intensity of staining for HRP, scaled from 1-3, was determined in 18 serial sections, centered over the impact site. Within each cross section, 11 regions of the spinal cord, indicated in the schematic drawing, were evaluated. The maximal score, indicative of intense HRP reactivity for any given section, was scored 33. The most pronounced staining for HRP occurred at the epicenter in all animals. There was a marked increase in permeability to HRP in the wild type mice as compared to either the MMP-nulls or wild type mice treated with GM6001.

FIGS. 7A and 7B illustrate the effect of blocking MMPs on permeability to luciferase after spinal cord injury. Abnormal permeability to luciferase was quantified in the epicenter at 24 hours post injury in MMP-9 null (N=6) and wild type littermates (N=5) (FIG. 7A) and in mice treated with vehicle (N=4) or GM6001 (begun at 3 hours after injury, N=6) (FIG. 7B). There is a significant reduction in barrier permeability in the MMP-9 null as compared to the wild type littermates (P=0. 02) and in drug treated as compared to vehicle controls (P=0.04). Values are the means ± S.D.

FIG. 8, panels A-C illustrates the effect of blocking MMPs on recruitment of inflammatory neutrophils into lesions after spinal cord injury. There appear to be greater numbers of neutrophils, as indicated with chloroacetate esterase staining in the spinal cord injured wild type (panel A) as compared to the MMP-9 null (panel B) animal at 24 hours post injury. The numbers of neutrophils were quantified within the epicenter of spinal cord injured mice treated with either GM6001 (N=4) or vehicle (N=4) at 24 hours post injury (panel C). There is a significant reduction in the numbers of neutrophils in mice treated with GM6001, as compared to vehicle controls (P=0.01). Values represent the means ± S.D. Panels A, B Scale bar=100 μm.

FIGS. 9A and 9B illustrate the effect of blocking MMPs on locomotor activity after spinal cord injury. Locomotor recovery was evaluated over a 6-week period, using a 21 point scale, in wild types (N=7) and MMP-9 nulls (N=7) (FIG. 9A), and in GM6001 (N=9) and vehicle treated (N=4) animals (FIG. 9B). Both the nulls and vehicle treated animals exhibited greater locomotor as compared to the their respective controls. Values represent the means ± S.D. * P=0.05, ** P<0.01.

FIG. 10, panels A-C illustrates the effect of blocking MMPs on preservation of spinal cord white matter. Typical appearance of residual white matter at 42 days post injury, as identified with a Luxol fast blue stain, in representative sections of GM6001 (panel A) and vehicle (panel B) treated animals. There is significantly greater preservation of white matter in the GM6001 treated as compared to the vehicle treated animals (panel C). Values represent the means ± S.D. P=0.01.

### DETAILED DESCRIPTION

This invention pertains to the discovery that that functional recovery after spinal cord injury is not simply the consequences of the initial mechanical destruction of tissue but is also attributed to the evolution of complex secondary events that contribute to early as well as delayed cell injury. In particular, this invention pertains to the discovery that proteinases, especially matrix metalloproteinases (MMPs), are mediators of early secondary vascular pathogenesis after spinal cord injury. Moreover, it is demonstrated herein, that inhibition of MMP expression and/or activity can improve recovery following trauma to neural tissue (e.g. spinal cord injury).

Matrix metalloproteinases (MMPs) are a family of extracellular zinc and calcium dependent endopeptidases (Birkedal-Hansen et al. (1993) Crit. Rev. Oral Biol. Med. 4:197-250) that degrade the extracellular matrix and other extracellular proteins (Sternlicht (1999) P. 503-603 In: Extracellular Matrix Proteinases. In: Guidebook to the Extracellular Matrix, Anchor and Adhesion Proteins (Kreis T, Vale R, eds), pp Oxford University Press, N.Y.; Sternlicht (2001) Annu Rev Cell Dev Biol., 17:463-516). MMPS are secreted in an inactive form, are typically activated in the extracellular matrix by a variety of mechanisms, and typically degrade the extracellular matrix. MMPs are essential for remodeling of the extracellular matrix, tissue morphogenesis, and wound healing (Werb, 1997). However, excessive proteolytic activity of MMPs can be detrimental, leading to numerous pathologic conditions including disruption of the blood-brain barrier (Rosenberg et al. (1994) Laboratory Investigation 71:417-422; Rosenberg et al. (1995) Brain Res., 703:151-155; Rosenberg and Navratil (1997) Neurology 48:921-926; Mun-Bryce and Rosenberg (1998) Am. J. Phpysiol., 274:R1203-1211; Rosenberg et al. (1998) Stroke 29:2189-2195; Yong et al. (2001) Nat Rev Neurosci 2:502-511; Mun-Bryce and Rosenberg (1998) Am. J. Phpysiol., 274:R1203-1211).

We have focused on the role of MMP-9 because of its established link to disruption of the blood-brain barrier, inflammation, and tissue injury. MMP-9 has been implicated in abnormal vascular permeability (Rosenberg et al. (1994) Laboratory Investigation 71:417-422; Rosenberg et al. (1995) Brain Res., 703:151-155; Mun-Bryce and Rosenberg (1998) Am. J. Phpysiol., 274:R1203-1211), associated with either hemorrhagic injury (Rosenberg et al. (1994) supra.) or inflammation (Mun-Bryce and Rosenberg (1998) supra.). Thus, abnormal increases in MMP-9 in both inflammatory cells as well as endothelial cells may collectively impair barrier function by degrading the vascular basement membrane. There is also evidence that MMP-9 increases in ischemic brain injury (Rosenberg et al. (1996) Neurology 46:1626-1632; Romanic et al. (1998) Stroke 29:1020-1030) and that the administration of a monoclonal antibody to MMP-9 reduces the hemispheric infarct size (Id.). Most recently, it has been shown that methylprednisolone, the only therapeutic agent approved by the Food and Drug Administration, suppresses the expression of MMP-9 after spinal cord injury (Xu et al. (2001) J. Neurosci., 21:92-97).

Our studies have identified matrix metalloproteinases (e.g. MMP-9) as significant contributors to blood-spinal cord barrier disruption and inflammation after spinal cord (or other neurological) injury and to impaired recovery after such damage.

MMP-9 is a member of the subclass of MMPs that includes MMP-2 (72-kD gelatinase/gelatinase A) and is known for degrading gelatin (denatured collagens), collagen IV, V, XI, elastin, and vitronectin. This protease is predominantly expressed by inflammatory cells including macrophages, lymphocytes, and neutrophils as well as endothelial cells. Recent studies suggest that MMP-9 inactivates alpha 1-antitrypsin, the primary physiologic inhibitor of leukocyte elastase, a step that is central to leukocyte migration.

In the central nervous system (CNS), there is a low constitutive expression of MMP-9 in microglia, astrocytes and hippocampal neurons, and it can be induced in astrocytes, microglia/macrophages and hippocampal cells.

Spinal cord injury (like other neurological injury) involves a primary physical injury as well as a secondary wave of inflammation and cell death that starts within hours of the primary injury and continues for days to weeks following injury. Much of the cell death that occurs in this secondary injury is common to many types of spinal cord injury and affords significant opportunity for pharmacological intervention to salvage spinal cord (or other neurological) function.

One major aspect of this secondary injury is infiltration of immune cells into the wound, and the secretion of many tissue degrading enzymes by these cells, including the matrix metalloproteinases.

We have discovered that matrix metalloproteinases, in particular the matrix metalloproteinase 9 (MMP-9, gelatinase B) are noticeably increased in experimental brain injury and is linked to opening of the blood-brain barrier. MMP-9 activity is also increased in such diseases as multiple sclerosis and amyotrophic lateral sclerosis, diseases that involve inflammation and demyelination of axons. MMP-9 was recently shown to cleave myelin basic protein, so it may contribute to demyelination of axons in spinal cord injury. Without being bound to a particular theory, we believe matrix metalloproteinases contribute to spinal cord injury (and injury associated in general with trauma to nervous tissue), and modulation (e.g. inhibition) of matrix metalloproteinase expression and/or activity facilitates recovery of function.

We have observed increased matrix metalloproteinase activity (e.g. MMP-9 activity) following spinal cord injury in the mouse, and have traced its activity and location throughout the wound healing process. To determine the contribution of MMP-9 activity to pathological processes (cell death, blood-spinal cord barrier disruption, and demyelination) as well as recovery processes, this injury model was applied to the MMP-9 null mouse. In a study of functional recovery from spinal cord injury, MMP-9 null mice demonstrated more rapid locomotor recovery than wild type mice.

We found that MMP-9 modulates the vascular response after spinal cord injury by promoting early inflammation and disruption of the blood-spinal cord barrier, which collective may contribute to cell injury. We then treated animals with an inhibitor of matrix metalloproteinases and found that animals, treated acutely (immediately or even after a short interval after spinal cord injury and for the next several days), exhibit significant recovery in locomotor ability similar to that observed in MMP-9 mutant (knockout) animals.

These results indicate that acute inhibition of expression or activity of matrix metalloproteinases has efficacy as a therapeutic strategy for the treatment of spinal cord injury (or other injury resulting from trauma to neurological tissue) in humans and non-human mammals.

Inhibition of expression or activity of matrix metalloproteinases can be accomplished by a number of methods. Such methods include, but are not limited to the use of matrix metalloprotease or metalloproteinase inhibitors (MMPIs) inhibitors (MMPIs), the use of MMP specific antisense molecules, the use of MMP specific ribozymes, intrabodies directed against MMPs, and the like. In cerain preferred embodiments, rapid inhibition of MMP activity is desired and consequently, in this context, MMPIs and MMPI antibodies are particularly preferred. In certain embodiments, inhibition of expression of MMP is effected by the use of an agent that is not a glucocorticoid. In a specific embodiment, inhibition of expression of MMP is effected by the use of an agent that is not methylprednisolone.

In certain embodiments, the use of matrix metalloproteinase inhibitors that specifically or non-specifically inhibit MMP-9 are utilized. We note that MMP-9 activity is linked to barrier disruption, inflammation, and tissue injury. In particular, MMP-9 has been implicated in barrier disruption associated with either hemorrhagic injury or inflammation. This protease cleaves a variety of proteins including, but not limited to extracellular matrix proteins comprising the basement membrane surrounding blood vessels. Thus, abnormal increases in MMP-9 in both inflammatory cells as well as endothelial cells may collectively impair barrier function by degrading the vascular basement membrane.. In one embodiment, the MMPI is a non-specific inhibitor of MMP-9. In another embodiment, the MMPI is a specific inhibitor of MMP-9. In specific embodiments, the MMP-9 specific inhibitor inhibits the the hydrolytic activity of MMP-9 by at least one times, two times, five times, 10 times, 20 times, 50 times, 100 times, or 500 times more than it inhibits other MMPs. In another embodiment, the inhibitor inhibits MMP-9, but does not inhibit MMP-2. In specific embodiments, the inhibitor inhibits the hydrolytic activity of MMP-9 by at least one times, two times, five times, 10 times, 20 times, 50 times, 100 times, or 500 times more than it inhibits MMP-2.

We believe matrix metalloproteinases (e.g. MMP-9) also increase in ischemic brain injury and that the administration of a neutralizing monoclonal antibody to MMP can reduces the hemispheric infarct size. The foregoing observations indicate that matrix metalloproteinases (e.g. MMP-9) contributes to blood- spinal cord barrier dysfunction after spinal cord injury and that modulation of MMPs after spinal cord injury stabilizes the barrier, limits inflammation, and promotes locomotor recovery. We believe the study described above provides the first evidence that MMPs play a key role in abnormal vascular permeability within the first 3 days after spinal cord injury and that blockade of MMPs during this critical period attenuates these vascular events and leads to improved locomotor recovery.

Thus, in a preferred embodiment, this invention provides a method of reducing neurological damage following trauma to nervous tissue in a mammal. The method preferably involves inhibiting activity or expression of a matrix metalloproteinase in the mammal before, during, or after the trauma. Typically one or more a matrix metalloproteinase inhibitor(s) will be administered to the mammal during or after the trauma typically in a concentration sufficient to reduce neurological damage and/or to improve recovery from the trauma..

Also provided is a method of reducing abnormal vascular permeability associated with spinal cord injury. This method involves inhibiting expression or activity of a matrix metalloproteinase (e.g. MMP-9), e.g. by administering to a mammal in need thereof one or more matrix metalloproteinase inhibitor(s) (MMPIs) in an amount sufficient to reduce abnormal vascular permeability associate with or following the spinal cord injury.

In still another embodiment, this invention provides a method of improving recovery of neurological function following injury to neurological tissue. his method involves inhibiting expression or activity of a matrix metalloproteinase (e.g. MMP-9), e.g by administering to a mammal in need thereof one or more matrix metalloproteinase inhibitor(s) (MMPIs) in an amount sufficient to improve recovery of neurological function following the injury.

Thus, in certain embodiments, the invention is readily practiced by administering to a mammal (e.g. a human, a non-human mammal) suffering from damage or trauma to neurological tissue (e.g. brain, spinal cord, etc.) or at risk of such trauma, an effective amount of a matrix metalloproteinase inhibitor (e.g. an MMP-9 inhibitor). In preferred embodiments, the MMPI(s) are administered at the time of trauma or shortly thereafter. In particular preferred embodiments, the administration is for up to 3 days after the trauma, preferably for up to 5 days, more preferably for up to 7 days, most preferably for up to 4 weeks, and most preferably until said administration produces no further beneficial effect.

I. Preferred Indications.

Agents that inhibit expression or activity of MMPs, especially of MMP-9 MMP-9 are believed to be effective and potent neuroprotective compounds, that can be used to reduce and prevent damage to a mammalian brain and/or spinal cord due to a number of following causes and etiologies including, but not limited to:

1) Physical trauma to the head or spinal cord (e.g., as can occur in automobile accidents, bad falls, sports injuries, etc.);

2) A brain concussion, which can occur due to physical trauma to the head, and in certain other types of situations involving rapid acceleration or deceleration of the head, stroke, including, but not limited to ischemic stroke caused by thrombosis or embolism, regardless of where a blood clot or other embolus originates in the body;

4) Other disruptions of proper blood flow through the brain, such as (i) cerebral hemorrhage; (ii) general circulatory failure or disruption, such as caused by cardiac arrest; (iii) hemodynamic shock, such as caused by loss of blood due to injury or hemorrhage elsewhere in the body; (iv) vasculatory damage, as can be caused by vascular disease, certain types of bacterial, viral, or other microbial infection, and other comparable causes; (v) cerebral or spinal tumors; and, (vi) glial cell swelling caused by infections (such as viral, bacterial, or other microbial meningitis, encephalitis, or encephalomyelitis, Reyes syndrome, or AIDS) or other mechanisms, such as hydrocephalus;

5) Hypoxic injury to the brain (i.e., inadequate oxygen supply), which arises as a direct result of any ischemic crisis, and which can also be caused by respiratory disruption, as occurs during incipient drowning or suffocation, carbon monoxide poisoning, etc.; and,

6) Post-operative brain injury or stress (e.g., as can be caused by neurosurgery, or by cardiopulmonary bypass for a prolonged period).

II. Matrix Metalloproteinase Inhibitors (MMPIs) to Inhibit MMP Expression or Activity.

In certain embodiments, this invention is readily practiced by administering to a mammal (e.g. a human, a non-human mammal) suffering from damage or trauma to neurological tissue (e.g. brain, spinal cord, etc.) or at risk of such trauma, an effective amount of a matrix metalloproteinase inhibitor (e.g. an MMP-9 inhibitor).

Numerous matrix metalloproteinase inhibitors are known, and all are believed to be useful in the methods of this invention. For example, 4-biarylbutyric and 5-biarylpentanoic acid derivatives are described in WO 96/15096. Other MMPIs include, but are not limited to Fenbufen and compounds related to fenbufen (see, e.e., U.S. Pat. No. 3,784,701; and Child, et al., (1977) J. Pharm. Sci., 66: 466-476), peptide MMPIs (see, e.g., U.S. Pat. Nos. 5,300,501; 5,530,128; 5,455,258; 5,552,419; WO 95/13289; and WO 96/11209), hydroxamic acids (see, e.g., U.S. Pat. Nos. 5,270,326, 5,530,161, 5,525,629, and 5,304,604), tricyclic butyric acid derivatives, biphenyl butyric acid derivatives, various tricyclic heteroaromatics (see, e.g., U.S. Pat. No. 6,350,885) heterocyclic substituted phenyl butyric acid derivatives (see, e.g., U.S. Pat. No. 6,265,432), sulfonamide derivatives, succinamide MMP inhibitors, sulfonated amino acid derivatives (see, e.g., WO 97/27174), anti-MMP antibodies (including full-length, fragments (e.g. Fab), single chain, etc.), and the like.

MMP compounds in clinical development include TIMP-2, marimastat, BAY-129566, CGS-27023A, BMS-275291 and metastat (COL-3), batimastat (BB-94), TIMP-1, prinomastat (AG-3340), RO 31-9790, and the like. MMP inhibitors that show specificity (preference) for MMP-9 include, but are not limited to BAY12-9566 and AG 33-40 (specific for MMP-1 and MMP-9).

In certain embodiments, the MMP inhibitors used in the methods of this invention include any one or more of the MMP inhibitors described in U.S. Pat. No. 5,948,780.

Other compounds that can inhibit the actions of matrix metalloproteinase enzymes can be identified utilizing routine in vitro and/or in vivo assays. One typical such assay measures the amount by which a test compound (candidate MMPI) reduces the hydrolysis of a thiopeptolide substrate caused by a matrix metalloproteinase enzyme. Such assays are described in detail by Ye et al. (1992) Biochemistry, 31(45): 11231-11235.

Thiopeptolide substrates show virtually no decomposition or hydrolysis in the absence of a matrix metalloproteinase enzyme. A typical thiopeptolide substrate commonly utilized for assays is Ac-Pro-Leu-Gly-thioester-Leu-Leu-Gly-O Et (SEQ ID NO:1). In certain embodiments, a 100 μL assay mixture will contain 50 mM of 2-morpholinoethane sulfonic acid monohydrate (MES, pH 6.0) 10 mM CaCl₂, 100 μM thiopeptolide substrate, and 1 mM 5,5′-dithio-bis-(2-nitro-benzoic acid) (DTNB). The thiopeptolide substrate concentration can be varied, e.g., from about 10 to about 800 μM to obtain Kₘ and K-values. The change in absorbance at 405 nm is monitored, e.g. on a microplate reader at a convenient temperature (e.g. room temperature ˜22° C.).

In a preferred embodiment, the calculation of the amount of hydrolysis of the thiopeptolide substrate is based on E₄₁₂=13600 m⁻¹ cm⁻¹ for the DTNB-derived product 3-carboxy-4-nitrothiophenoxide. Assays are carried out with and without test matrix metalloproteinase inhibitor compounds, or with test MMPI compounds at various concentrations), and the amount of hydrolysis is compared for a determination of inhibitory activity of the test compound.

III. Inhibition of MMP Expression.

In certain embodiments, this invention contemplates the use of agents and approaches other than the use of MMP inhibitors. Thus, for example, MMPI expression and/or activity can be inhibited by antisense molecules, MMP specific riibozymes, MMP specific catalytic DNAs, MMP specific RNAi, intrabodies directed against MMPs (e.g. MMP-9), and “gene therapy” approaches that knock out MMPs in particular target cells and/or tissues.

A) Antisense Approaches.

MMP expression can be downregulated or entirely inhibited by the use of antisense molecules. An “antisense sequence or antisense nucleic acid” is a nucleic acid that is complementary to the coding MMP mRNA nucleic acid sequence or a subsequence thereof. Binding of the antisense molecule to the MMP mRNA interferes with normal translation of the MMP polypeptide (e.g. MMP-9).

Thus, in accordance with certain embodiments of this invention, antisense molecules include oligonucleotides and oligonucleotide analogs that are hybridizable with MMP messenger RNA. This relationship is commonly denominated as “antisense.” The oligonucleotides and oligonucleotide analogs are able to inhibit the function of the RNA, either its translation into protein, its translocation into the cytoplasm, or any other activity necessary to its overall biological function. The failure of the messenger RNA to perform all or part of its function results in a reduction or complete inhibition of expression of the MMP polypeptides.

In the context of this invention, the term “oligonucleotide” refers to a polynucleotide formed from naturally-occurring bases and/or cyclofuranosyl groups joined by native phosphodiester bonds. This term effectively refers to naturally-occurring species or synthetic species formed from naturally-occurring subunits or their close homologs. The term “oligonucleotide” may also refer to moieties which function similarly to oligonucleotides, but which have non naturally-occuring portions. Thus, oligonucleotides may have altered sugar moieties or inter-sugar linkages. Exemplary among these are the phosphorothioate and other sulfur containing species that are known for use in the art. In accordance with some preferred embodiments, at least one of the phosphodiester bonds of the oligonucleotide has been substituted with a structure which functions to enhance the ability of the compositions to penetrate into the region of cells where the RNA whose activity is to be modulated is located. It is preferred that such substitutions comprise phosphorothioate bonds, methyl phosphonate bonds, or short chain alkyl or cycloalkyl structures. In accordance with other preferred embodiments, the phosphodiester bonds are substituted with structures which are, at once, substantially non-ionic and non-chiral, or with structures which are chiral and enantiomerically specific. Persons of ordinary skill in the art will be able to select other linkages for use in the practice of the invention.

In one embodiment, the internucleotide phosphodiester linkage is replaced with a peptide linkage. Such peptide nucleic acids tend to show improved stability, penetrate the cell more easily, and show enhances affinity for their target. Methods of making peptide nucleic acids are known to those of skill in the art (see, e.g., U.S. Pat. Nos. 6,015,887, 6,015,710, 5,986,053, 5,977,296, 5,902,786, 5,864,010, 5,786,461, 5,773,571, 5,766,855, 5,736,336, 5,719,262, and 5,714,331).

Oligonucleotides may also include species that include at least some modified base forms. Thus, purines and pyrimidines other than those normally found in nature may be so employed. Similarly, modifications on the furanosyl portions of the nucleotide subunits may also be effected, as long as the essential tenets of this invention are adhered to. Examples of such modifications are 2′-O-alkyl- and 2′-halogen-substituted nucleotides. Some specific examples of modifications at the 2′ position of sugar moieties which are useful in the present invention are OH, SH, SCH₃, F, OCH₃, OCN, O(CH₂)[n]NH₂ or O(CH₂)[n]CH₃, where n is from 1 to about 10, and other substituents having similar properties.

Such oligonucleotides are best described as being functionally interchangeable with natural oligonucleotides or synthesized oligonucleotides along natural lines, but which have one or more differences from natural structure. All such analogs are comprehended by this invention so long as they function effectively to hybridize with messenger RNA of MMP to inhibit the function of that RNA.

The oligonucleotides in accordance with certain embodiments of this invention comprise from about 3 to about 50 subunits. It is more preferred that such oligonucleotides and analogs comprise from about 8 to about 25 subunits and still more preferred to have from about 12 to about 20 subunits. As will be appreciated, a subunit is a base and sugar combination suitably bound to adjacent subunits through phosphodiester or other bonds. The oligonucleotides used in accordance with this invention can be conveniently and routinely made through the well-known technique of solid phase synthesis. Equipment for such syntheses is sold by several vendors (e.g. Applied Biosystems). Any other means for such synthesis may also be employed, however, the actual synthesis of the oligonucleotides is well within the talents of the routineer. It is also will known to prepare other oligonucleotide such as phosphorothioates and alkylated derivatives.

B) Catalytic RNAs and DNAs

1) Ribozymes.

In another approach, MMP (e.g., MMP-9) expression can be inhibited by the use of ribozymes. As used herein, “ribozymes” include RNA molecules that contain antisense sequences for specific recognition, and an RNA-cleaving enzymatic activity. The catalytic strand cleaves a specific site in a target (MMP) RNA, preferably at greater than stoichiometric concentration. Two “types” of ribozymes are particularly useful in this invention, the hammerhead ribozyme (Rossi et al. (1991) Pharmac. Ther. 50: 245-254) and the hairpin ribozyme (Hampel et al. (1990) Nucl. Acids Res. 18: 299-304, and U.S. Pat. No. 5,254,678).

Because both hammerhead and hairpin ribozymes are catalytic molecules having antisense and endoribonucleotidase activity, ribozyme technology has emerged as a potentially powerful extension of the antisense approach to gene inactivation. The ribozymes of the invention typically consist of RNA, but such ribozymes may also be composed of nucleic acid molecules comprising chimeric nucleic acid sequences (such as DNA/RNA sequences) and/or nucleic acid analogs (e.g., phosphorothioates).

Accordingly, within one aspect of the present invention ribozymes have the ability to inhibit MMP expression. Such ribozymes may be in the form of a “hammerhead” (for example, as described by Forster and Symons (1987) Cell 48: 211-220,; Haseloff and Gerlach (1988) Nature 328: 596-600; Walbot and Bruening (1988) Nature 334: 196; Haseloff and Gerlach (1988) Nature 334: 585) or a “hairpin” (see, e.g. U.S. Pat. No. 5,254,678 and Hampel et al., European Patent Publication No. 0 360 257, published Mar. 26, 1990), and have the ability to specifically target, cleave and MMP nucleic acids.

The ribozymes for this invention, as well as DNA encoding such ribozymes and other suitable nucleic acid molecules can be chemically synthesized using methods well known in the art for the synthesis of nucleic acid molecules. Alternatively, Promega, Madison, Wis., USA, provides a series of protocols suitable for the production of RNA molecules such as ribozymes. The ribozymes also can be prepared from a DNA molecule or other nucleic acid molecule (which, upon transcription, yields an RNA molecule) operably linked to an RNA polymerase promoter, e.g., the promoter for T7 RNA polymerase or SP6 RNA polymerase. Such a construct may be referred to as a vector. Accordingly, also provided by this invention are nucleic acid molecules, e.g., DNA or cDNA, coding for the ribozymes of this invention. When the vector also contains an RNA polymerase promoter operably linked to the DNA molecule, the ribozyme can be produced in vitro upon incubation with the RNA polymerase and appropriate nucleotides. In a separate embodiment, the DNA may be inserted into an expression cassette (see, e.g., Cotten and Birnstiel (1989) EMBO J 8(12):3861-3866; Hempel et al. (1989) Biochem. 28: 4929-4933, etc.).

After synthesis, the ribozyme can be modified by ligation to a DNA molecule having the ability to stabilize the ribozyme and make it resistant to RNase. Alternatively, the ribozyme can be modified to the phosphothio analog for use in liposome delivery systems. This modification also renders the ribozyme resistant to endonuclease activity.

The ribozyme molecule also can be in a host prokaryotic or eukaryotic cell in culture or in the cells of an organism/patient. Appropriate prokaryotic and eukaryotic cells can be transfected with an appropriate transfer vector containing the DNA molecule encoding a ribozyme of this invention. Alternatively, the ribozyme molecule, including nucleic acid molecules encoding the ribozyme, may be introduced into the host cell using traditional methods such as transformation using calcium phosphate precipitation (Dubensky et al. (1984) Proc. Natl. Acad. Sci., USA, 81: 7529-7533), direct microinjection of such nucleic acid molecules into intact target cells (Acsadi et al. (1991) Nature 352: 815-818), and electroporation whereby cells suspended in a conducting solution are subjected to an intense electric field in order to transiently polarize the membrane, allowing entry of the nucleic acid molecules. Other procedures include the use of nucleic acid molecules linked to an inactive adenovirus (Cotton et al. (1990) Proc. Natl. Acad. Sci., USA, 89 :6094), lipofection (Felgner et al. (1989) Proc. Natl. Acad. Sci. USA 84: 7413-7417), microprojectile bombardment (Williams et al. (1991) Proc. Natl. Acad. Sci., USA, 88: 2726-2730), polycation compounds such as polylysine, receptor specific ligands, liposomes entrapping the nucleic acid molecules, spheroplast fusion whereby E coli containing the nucleic acid molecules are stripped of their outer cell walls and fused to animal cells using polyethylene glycol, viral transduction, (Cline et al., (1985) Pharmac. Ther. 29: 69; and Friedmann et al. (1989) Science 244: 1275), and DNA ligand (Wu et al (1989) J Biol. Chem. 264: 16985-16987), as well as psoralen inactivated viruses such as Sendai or Adenovirus. In one preferred embodiment, the ribozyme is introduced into the host cell utilizing a lipid, a liposome or a retroviral vector.

When the DNA molecule is operatively linked to a promoter for RNA transcription, the RNA can be produced in the host cell when the host cell is grown under suitable conditions favoring transcription of the DNA molecule. The vector can be, but is not limited to, a plasmid, a virus, a retrotransposon or a cosmid. Examples of such vectors are disclosed in U.S. Pat. No. 5,166,320. Other representative vectors include, but are not limited to adenoviral vectors (e.g., WO 94/26914, WO 93/9191; Kolls et al. (1994) PNAS 91(1):215-219; Kass-Eisler et al., (1993) Proc. Natl. Acad. Sci., USA, 90(24): 11498-502, Guzman et al. (1993) Circulation 88(6): 2838-48, 1993; Guzman et al. (1993) Cir. Res. 73(6):1202-1207, 1993; Zabner et al. (1993) Cell 75(2): 207-216; Li et al. (1993) Hum Gene Ther. 4(4): 403-409; Caillaud et al. (1993) Eur. J Neurosci. 5(10): 1287-1291), adeno-associated vector type 1 (“AAV-1”) or adeno-associated vector type 2 (“AAV-2”) (see WO 95/13365; Flotte et al. (1993) Proc. Natl. Acad. Sci., USA, 90(22):10613-10617), retroviral vectors (e.g., EP 0 415 731; WO 90/07936; WO 91/02805; WO 94/03622; WO 93/25698; WO 93/25234; U.S. Pat. No. 5,219,740; WO 93/11230; WO 93/10218) and herpes viral vectors (e.g., U.S. Pat. No. 5,288,641). Methods of utilizing such vectors in gene therapy are well known in the art, see, for example, Larrick and Burck (1991) Gene Therapy: Application of Molecular Biology, Elsevier Science Publishing Co., Inc., New York, N.Y., and Kreigler (1990) Gene Transfer and Expression: A Laboratory Manual, W. H. Freeman and Company, New York.

To produce ribozymes in vivo utilizing vectors, the nucleotide sequences coding for ribozymes are preferably placed under the control of a strong promoter such as the lac, SV40 late, SV40 early, or lambda promoters. Ribozymes are then produced directly from the transfer vector in vivo

2) Catalytic DNA

In a manner analogous to ribozymes, DNAs are also capable of demonstrating catalytic (e.g. nuclease) activity. While no such naturally-occurring DNAs are known, highly catalytic species have been developed by directed evolution and selection. Beginning with a population of 10¹⁴ DNAs containing 50 random nucleotides, successive rounds of selective amplification, enriched for individuals that best promote the Pb²⁺-dependent cleavage of a target ribonucleoside 3′-O—P bond embedded within an otherwise all-DNA sequence. By the fifth round, the population as a whole carried out this reaction at a rate of 0.2 min⁻¹. Based on the sequence of 20 individuals isolated from this population, a simplified version of the catalytic domain that operates in an intermolecular context with a turnover rate of 1 min⁻¹ (see, e.g., Breaker and Joyce (1994) Chem Biol 4: 223-229.

In later work, using a similar strategy, a DNA enzyme was made that could cleave almost any targeted RNA substrate under simulated physiological conditions. The enzyme is comprised of a catalytic domain of 15 deoxynucleotides, flanked by two substrate-recognition domains of seven to eight deoxynucleotides each. The RNA substrate is bound through Watson-Crick base pairing and is cleaved at a particular phosphodiester located between an unpaired purine and a paired pyrimidine residue. Despite its small size, the DNA enzyme has a catalytic efficiency (kcat/Km) of approximately 10⁹ M⁻¹ min⁻¹ under multiple turnover conditions, exceeding that of any other known nucleic acid enzyme. By changing the sequence of the substrate-recognition domains, the DNA enzyme can be made to target different RNA substrates (Santoro and Joyce (1997) Proc. Natl. Acad. Sci., USA, 94(9): 4262-4266). Modifying the appropriate targeting sequences (e.g. as described by Santoro and Joyce, supra.) the DNA enzyme can easily be retargeted to MMP mRNA thereby acting like a ribozyme.

C) RNAi Inhibition of MMP Expression.

Post-transcriptional gene silencing (PTGS) or RNA interference (RNAi) refers to a mechanism by which double-stranded (sense strand) RNA (dsRNA) specifically blocks expression of its homologous gene when injected, or otherwise introduced into cells. The discovery of this incidence came with the observation that injection of antisense or sense RNA strands into Caenorhabditis elegans cells resulted in gene-specific inactivation (Guo and Kempheus (1995) Cell 81: 611-620). While gene inactivation by the antisense strand was expected, gene silencing by the sense strand came as a surprise. Adding to the surprise was the finding that this gene-specific inactivation actually came from trace amounts of contaminating dsRNA (Fire et al. (1998) Nature 391: 806-811).

Since then, this mode of post-transcriptional gene silencing has been tied to a wide variety of organisms: plants, flies, trypanosomes, planaria, hydra, zebrafish, and mice (Zamore et al. (2000). Cell 101: 25-33; Gura (2000) Nature 404: 804-808). RNAi activity has been associated with functions as disparate as transposon-silencing, anti-viral defense mechanisms, and gene regulation (Grant (1999) Cell 96: 303-306).

By injecting dsRNA into tissues, one can inactivate specific genes not only in those tissues, but also during various stages of development. This is in contrast to tissue-specific knockouts or tissue-specific dominant-negative gene expressions, which do not allow for gene silencing during various stages of the developmental process (Gura (2000) Nature 404: 804-808). The double-stranded RNA is cut by a nuclease activity into 21-23 nucleotide fragments. These fragments, in turn, target the homologous region of their corresponding mRNA, hybridize, and result in a double-stranded substrate for a nuclease that degrades it into fragments of the same size (Hammond et al. (2000) Nature, 404: 293-298; Zamore et al. (2000). Cell 101: 25-33).

Double stranded RNA (dsRNA) can be introduced into cells by any of a wide variety of means. Such methods include, but are not limited to lipid-mediated transfection (e.g. using reagents such as lipofectamine), liposome delivery, dendrimer-mediated transfection, and gene transfer using a viral or bacterial vector. Where the vector expresses (transcribes) a single-stranded RNA, the vector can be designed to transcribe two complementary RNA strands that will then hybridize to form a double-stranded RNA.

D) Intrabodies.

In still another embodiment, MMP expression/activity can be inhibited by transfecting the subject cell(s) (e.g., cells of the vascular endothelium) with a nucleic acid construct that expresses an intrabody. An intrabody is an intracellular antibody, in this case, capable of recognizing and binding to a MMP polypeptide. The intrabody is expressed by an “antibody cassette”, containing a sufficient number of nucleotides coding for the portion of an antibody capable of binding to the target (MMP polypeptide) operably linked to a promoter that will permit expression of the antibody in the cell(s) of interest. The construct encoding the intrabody is delivered to the cell where the antibody is expressed intracellularly and binds to the target MMP, thereby disrupting the target from its normal action. This antibody is sometimes referred to as an “intrabody”.

In one preferred embodiment, the “intrabody gene” (antibody) of the antibody cassette would utilize a cDNA, encoding heavy chain variable (V_{H}) and light chain variable (V_{L}) domains of an antibody which can be connected at the DNA level by an appropriate oligonucleotide as a bridge of the two variable domains, which on translation, form a single peptide (referred to as a single chain variable fragment, “sFv”) capable of binding to a target such as an MMP protein. The intrabody gene preferably does not encode an operable secretory sequence and thus the expressed antibody remains within the cell.

Anti-MMP antibodies suitable for use/expression as intrabodies in the methods of this invention can be readily produced by a variety of methods. Such methods include, but are not limited to, traditional methods of raising “whole” polyclonal antibodies, which can be modified to form single chain antibodies, or screening of, e.g. phage display libraries to select for antibodies showing high specificity and/or avidity for MMP. Such screening methods are described above in some detail.

The antibody cassette is delivered to the cell by any of the known means. One preferred delivery system is described in U.S. Pat. No. 6,004,940. Methods of making and using intrabodies are described in detail in U.S. Pat. Nos. 6,072,036, 6,004,940, and 5,965,371.

E) MMP Antibodies.

In certain embodiments, this invention contemplates the use of MMP specific (neutralizing) antibodies (e.g. anti-MMP-9) antibodies to specifically inhibit activity of the MMP(s). Such anti-MMP antibodies include, but are not limited to full size antibodies, antibody fragments, and single chain antibodies. In various embodiments, the antibodies are chimeric or humanized, or human antibodies.

As indicated above, the MMP-neutralizing antibodies of this invention can be administered to an organism (e.g., a human patient) for therapeutic purposes (e.g., prophylaxis and/or mitigation of injury associated with trauma to neurological tissue). Antibodies administered to an organism other than the species in which they are raised can be immunogenic. Thus, for example, murine antibodies repeatedly administered to a human often induce an immunologic response against the antibody (e.g., the human anti-mouse antibody (HAMA) response). While this is typically not a problem for the use of non-human antibodies of this invention as they are typically not utilized repeatedly, the immunogenic properties of the antibody are reduced by altering portions, or all, of the antibody into characteristically human sequences thereby producing chimeric or human antibodies, respectively.

1) Humanized (Chimeric) Antibodies.

Humanized (chimeric) antibodies are immunoglobulin molecules comprising a human and non-human portion. More specifically, the antigen combining region (or variable region) of a humanized chimeric antibody is derived from a non-human source (e.g., murine) and the constant region of the chimeric antibody (which confers biological effector function to the immunoglobulin) is derived from a human source. The humanized chimeric antibody should have the antigen binding specificity of the non-human antibody molecule and the effector function conferred by the human antibody molecule. A large number of methods of generating chimeric antibodies are well known to those of skill in the art (see, e.g., U.S. Pat. Nos: 5,502,167, 5,500,362, 5,491,088, 5,482,856, 5,472,693, 5,354,847, 5,292,867, 5,231,026, 5,204,244, 5,202,238, 5,169,939, 5,081,235, 5,075,431, and 4,975,369).

Chimeric antibodies comprising one or more CDRs from human species and framework regions from a non-human immunoglobulin molecule can be produced using a variety of techniques known in the art including, for example, CDR-grafting (EP 239,400; PCT publication No. WO 91/09967; and U.S. Pat. Nos. 5,225,539, 5,530,101, and 5,585,089), veneering or resurfacing (EP 592,106; EP 519,596; Padlan (1991) Molecular Immunology 28(4/5): 489-498; Studnicka et al. (1994) Protein Engineering 7(6): 805-814; and Roguska et al. (1994) Proc. Natl. Acad. Sci., USA, 91: 969-973), and chain shuffling (U.S. Pat. No. 5,565,332). Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions (see, e.g., U.S. Pat. No. 5,585,089; and Riechmann et al. (1988) Nature 332: 323).

In general, the procedures used to produce chimeric antibodies consist of the following steps (the order of some steps may be interchanged): (a) identifying and cloning the correct gene segment encoding the antigen binding portion of the antibody molecule; this gene segment (known as the VDJ, variable, diversity and joining regions for heavy chains or VJ, variable, joining regions for light chains (or simply as the V or variable region) may be in either the cDNA or genomic form; (b) cloning the gene segments encoding the constant region or desired part thereof; (c) ligating the variable region to the constant region so that the complete chimeric antibody is encoded in a transcribable and translatable form; (d) ligating this construct into a vector containing a selectable marker and gene control regions such as promoters, enhancers and poly(A) addition signals; (e) amplifying this construct in a host cell (e.g., bacteria); (f) introducing the DNA into eukaryotic cells (transfection) most often mammalian lymphocytes; and culturing the host cell under conditions suitable for expression of the chimeric antibody.

Antibodies of several distinct antigen binding specificities have been manipulated by these protocols to produce chimeric proteins (e.g., anti-TNP: Boulianne et al. (1984) Nature, 312: 643; and anti-tumor antigens: Sahagan et al. (1986) J. Immunol., 137: 1066). Likewise several different effector functions have been achieved by linking new sequences to those encoding the antigen binding region. Some of these include enzymes (Neuberger et al. (1984) Nature 312: 604), immunoglobulin constant regions from another species and constant regions of another immunoglobulin chain (Sharon et al. (1984) Nature 309: 364; Tan et al., (1985) J. Immunol. 135: 3565-3567).

In one preferred embodiment, a recombinant DNA vector is used to transfect a cell line that produces a MMP-neutralizing antibody. The novel recombinant DNA vector contains a “replacement gene” to replace all or a portion of the gene encoding the immunoglobulin constant region in the cell line (e.g., a replacement gene may encode all or a portion of a constant region of a human immunoglobulin, a specific immunoglobulin class, or an enzyme, a toxin, a biologically active peptide, a growth factor, inhibitor, or a linker peptide to facilitate conjugation to a drug, toxin, or other molecule, etc.), and a “target sequence” which allows for targeted homologous recombination with immunoglobulin sequences within the antibody producing cell.

In another embodiment, a recombinant DNA vector is used to transfect a cell line that produces an antibody having a desired effector function, (e.g., a constant region of a human immunoglobulin) in which case, the replacement gene contained in the recombinant vector may encode all or a portion of a region of an MMP-neutralizing antibody and the target sequence contained in the recombinant vector allows for homologous recombination and targeted gene modification within the antibody producing cell. In either embodiment, when only a portion of the variable or constant region is replaced, the resulting chimeric antibody may define the same antigen and/or have the same effector function yet be altered or improved so that the chimeric antibody may demonstrate a greater antigen specificity, greater affinity binding constant, increased effector function, or increased secretion and production by the transfected antibody producing cell line, etc.

Regardless of the embodiment practiced, the processes of selection for integrated DNA (via a selectable marker), screening for chimeric antibody production, and cell cloning, can be used to obtain a clone of cells producing the chimeric antibody.

Thus, a piece of DNA which encodes a modification for a monoclonal antibody can be targeted directly to the site of the expressed immunoglobulin gene within a B-cell or hybridoma cell line. DNA constructs for any particular modification may be used to alter the protein product of any monoclonal cell line or hybridoma. Such a procedure circumvents the costly and time consuming task of cloning both heavy and light chain variable region genes from each B-cell clone expressing a useful antigen specificity. In addition to circumventing the process of cloning variable region genes, the level of expression of chimeric antibody should be higher when the gene is at its natural chromosomal location rather than at a random position. Detailed methods for preparation of chimeric (humanized) antibodies can be found in U.S. Pat. No. 5,482,856.

2) Human Antibodies.

In another embodiment, this invention provides for fully human anti-MMP-neutralizing antibodies. Human antibodies consist entirely of characteristically human polypeptide sequences. The human MMP-neutralizing antibodies of this invention can be produced in using a wide variety of methods (see, e.g., Larrick et al., U.S. Pat. No. 5,001,065, for review).

In one preferred embodiment, fully human antibodies are produced using phage display methods as described herein. However, instead of utilizing a murine gene library, a human gene library is used. Methods of producing fully human gene libraries are well known to those of skill in the art (see, e.g., Vaughn et al. (1996) Nature Biotechnology, 14(3): 309-314, Marks et al. (1991) J. Mol. Biol., 222: 581-597, and PCT/US96/10287).

In another preferred embodiment, the human MMP-neutralizing antibodies of the present invention are usually initially in trioma cells. Genes encoding the antibodies are then cloned and expressed in other cells, particularly, nonhuman mammalian cells.

The general approach for producing human antibodies by trioma technology has been described by Ostberg et al. (1983) Hybridoma 2: 361-367, Ostberg, U.S. Pat. No. 4,634,664, and Engelman et al., U.S. Pat. No. 4,634,666. The antibody-producing cell lines obtained by this method are called triomas because they are descended from three cells; two human and one mouse. Triomas have been found to produce antibody more stably than ordinary hybridomas made from human cells.

Preparation of trioma cells requires an initial fusion of a mouse myeloma cell line with unimmunized human peripheral B lymphocytes. This fusion generates a xenogenic hybrid cell containing both human and mouse chromosomes (see, Engelman, supra.). Xenogenic cells that have lost the capacity to secrete antibodies are selected. Preferably, a xenogenic cell is selected that is resistant to 8-azaguanine. Such cells are unable to propagate on hypoxanthine-aminopterin-thymidine (HAT) or azaserin-hypoxanthine (AH) media.

The capacity to secrete antibodies is conferred by a further fusion between the xenogenic cell and B-lymphocytes immunized against an MMP polypeptide (e.g., MMP-9, MMP-9 subsequences, etc.). The B-lymphocytes are obtained from the spleen, blood or lymph nodes of human donor. If antibodies against a specific antigen or epitope are desired, it is preferable to use that antigen or epitope thereof as the immunogen rather than the entire polypeptide. Alternatively, B-lymphocytes are obtained from an unimmunized individual and stimulated with a MMP polypeptide, or a epitope thereof, in vitro. In a further variation, B-lymphocytes are obtained from an infected, or otherwise immunized individual, and then hyperimmunized by exposure to an MMP polypeptide for about seven to fourteen days, in vitro.

The immunized B-lymphocytes prepared by one of the above procedures are fused with a xenogenic hybrid cell by well known methods. For example, the cells are treated with 40-50% polyethylene glycol of MW 1000-4000, at about 37° C. for about 5-10 min. Cells are separated from the fusion mixture and propagated in media selective for the desired hybrids. When the xenogenic hybrid cell is resistant to 8-azaguanine, immortalized trioma cells are conveniently selected by successive passage of cells on HAT or AH medium. Other selective procedures are, of course, possible depending on the nature of the cells used in fusion. Clones secreting antibodies having the required binding specificity are identified by assaying the trioma culture medium for the ability to bind to the MMP polypeptide or an epitope thereof. Triomas producing human antibodies having the desired specificity are subcloned by the limiting dilution technique and grown in vitro in culture medium, or are injected into selected host animals and grown in vivo.

The trioma cell lines obtained are then tested for the ability to bind a MMP polypeptide or an epitope thereof. Antibodies are separated from the resulting culture medium or body fluids by conventional antibody-fractionation procedures, such as ammonium sulfate precipitation, DEAE cellulose chromatography and affinity chromatography.

Although triomas are genetically stable they do not produce antibodies at very high levels. Expression levels can be increased by cloning antibody genes from the trioma into one or more expression vectors, and transforming the vector into a cell line such as the cell lines typically used for expression of recombinant or humanized immunoglobulins. As well as increasing yield of antibody, this strategy offers the additional advantage that immunoglobulins are obtained from a cell line that does not have a human component, and does not therefore need to be subjected to the especially extensive viral screening required for human cell lines.

The genes encoding the heavy and light chains of immunoglobulins secreted by trioma cell lines are cloned according to methods, including but not limited to, the polymerase chain reaction (PCR), known in the art (see, e.g., Sambrook et al., Molecular Cloning: A Laboratory Manual, 2nd ed., Cold Spring Harbor, N.Y., 1989; Berger & Kimmel, Methods in Enzymology, Vol. 152: Guide to Molecular Cloning Techniques, Academic Press, Inc., San Diego, Calif., 1987; Co et al. (1992) J. Immunol., 148: 1149). For example, genes encoding heavy and light chains are cloned from a trioma's genomic DNA or cDNA produced by reverse transcription of the trioma's RNA. Cloning is accomplished by conventional techniques including the use of PCR primers that hybridize to the sequences flanking or overlapping the genes, or segments of genes, to be cloned.

Typically, recombinant constructs comprise DNA segments encoding a complete human immunoglobulin heavy chain and/or a complete human immunoglobulin light chain of an immunoglobulin expressed by a trioma cell line. Alternatively, DNA segments encoding only a portion of the primary antibody genes are produced, which portions possess binding and/or effector activities. Other recombinant constructs contain segments of trioma cell line immunoglobulin genes fused to segments of other immunoglobulin genes, particularly segments of other human constant region sequences (heavy and/or light chain). Human constant region sequences can be selected from various reference sources, including but not limited to those listed in Kabat et al. (1987) Sequences of Proteins of Immunological Interest, U.S. Department of Health and Human Services.

In addition to the DNA segments encoding MMP-neutralizing immunoglobulins or fragments thereof, other substantially homologous modified immunoglobulins can be readily designed and manufactured utilizing various recombinant DNA techniques known to those skilled in the art such as site-directed mutagenesis (see Gillman & Smith (1979) Gene, 8: 81-97; Roberts et al. (1987) Nature 328: 731-734). Such modified segments will usually retain antigen binding capacity and/or effector function. Moreover, the modified segments are usually not so far changed from the original trioma genomic sequences to prevent hybridization to these sequences under stringent conditions. Because, like many genes, immunoglobulin genes contain separate functional regions, each having one or more distinct biological activities, the genes may be fused to functional regions from other genes to produce fusion proteins (e.g., immunotoxins) having novel properties or novel combinations of properties.

The genomic sequences can be cloned and expressed according to standard methods as described herein.

Other approaches to antibody production include in vitro immunization of human blood. In this approach, human blood lymphocytes capable of producing human antibodies are produced. Human peripheral blood is collected from the patient and is treated to recover mononuclear cells. The suppressor T-cells then are removed and remaining cells are suspended in a tissue culture medium to which is added the antigen and autologous serum and, preferably, a nonspecific lymphocyte activator. The cells then are incubated for a period of time so that they produce the specific antibody desired. The cells then can be fused to human myeloma cells to immortalize the cell line, thereby to permit continuous production of antibody (see U.S. Pat. No. 4,716,111).

In another approach, mouse-human hybridomas which produces human MMP-neutralizing antibodies are prepared (see, e.g., U.S. Pat. No. 5,506,132). Other approaches include immunization of murines transformed to express human immunoglobulin genes, and phage display screening (Vaughan et al. supra.).

F) Small Organic Molecules.

In still another embodiment, MMP expression and/or MMP protein activity can be inhibited by the use of small organic molecules. Such molecules include, but are not limited to molecules that specifically bind to the DNA comprising the MMP promoter and/or coding region, molecules that bind to and complex with MMP mRNA, molecules that inhibit the signaling pathway that results in MMP upregulation, and molecules that bind to and/or compete with MMP polypeptides (e.g. the various MMPIs described above). Small organic molecules effective at inhibiting MMP expression or activity can be identified with routine screening using the methods described herein.

The methods of inhibiting MMP expression described above are meant to be illustrative and not limiting. In view of the teachings provided herein, other methods of inhibiting MMP will be known to those of skill in the art.

IV. Modes of Administration.

The mode of administration of the MMP blocking agent (agent that inhibits expression and/or activity of one or more MMPs) depends on the nature of the particular agent. Antisense molecules, catalytic RNAs (ribozymes), catalytic DNAs, small organic molecules, RNAi, and other molecules (e.g. lipids, antibodies, etc.) used as MMP inhibitors can be formulated as pharmaceuticals (e.g. with suitable excipient) and delivered using standard pharmaceutical formulation and delivery methods as described below. Antisense molecules, catalytic RNAs (ribozymes), catalytic DNAs, and additionally, knockout constructs, and constructs encoding intrabodies can be delivered and (if necessary) expressed in target cells (e.g. vascular endothelial cells) using methods of gene therapy, e.g. as described below.

A) Pharmaceutical Formulations.

The compositions of the invention include bulk drug compositions useful in the manufacture of non-pharmaceutical compositions (e.g., impure or non-sterile compositions) and pharmaceutical compositions (i.e., compositions that are suitable for administration to a subject or patient) that can be used directly and/or in the preparation of unit dosage forms. Such compositions comprise a therapeutically effective amount of one or more therapeutic agents (e.g. MMPIs) disclosed herein or a combination of the agent(s) and a pharmaceutically acceptable carrier. Preferably, compositions of the invention comprise a therapeutically effective amount of an inhibitor of expression and/or activity of an MMP (e.g. an inhibitor of MMP-9), and a, optionally, a pharmaceutically acceptable carrier.

The agents that inhibit expression or activity of MMPIs (e.g., matrix metalloproteinase inhibitors (MMPIs)) used in the methods of this invention, (e.g. to reduce neurological injury) can be prepared and administered in a wide variety of rectal, oral and parenteral dosage forms for treating and preventing neurological damage, increased vascular permeability associated with trauma, and the like. One or more NMPIs can be administered by injection, that is, intravenously, intramuscularly, intracutaneously, subcutaneously, intraduodenally, or intraperitoneally. Also, the compounds can be administered by inhalation, for example, intranasally. Additionally, the compounds can be administered transdermally.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans, or suitable for administration to an animal or human. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of the compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Pharmaceutical compositions comprising the inhibitors of MMP expression and/or activity can be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries that facilitate processing of the molecules into preparations that can be used pharmaceutically. Proper formulation is dependent upon the route of administration chosen.

For topical or transdermal administration, the inhibitors of MMP expression and/or activity can be formulated as solutions, gels, ointments, creams, lotion, emulsion, suspensions, etc. as are well-known in the art. Systemic formulations include those designed for administration by injection, e.g. subcutaneous, intravenous, intramuscular, intrathecal or intraperitoneal injection, as well as those designed for transdermal, transmucosal, inhalation, oral or pulmonary administration.

For injection, the inhibitors of MMP expression and/or activity can be formulated in aqueous solutions, preferably in physiologically compatible buffers such as Hanks's solution, Ringer's solution, or physiological saline buffer. The solution can contain formulatory agents such as suspending, stabilizing and/or dispersing agents. Alternatively, compositions comprising the inhibitors of MMP expression and/or activity can be in powder form for constitution with a suitable vehicle, e.g., sterile pyrogen-free water, before use.

For transmucosal administration, penetrants appropriate to the barrier to be permeated are used in the formulation. Such penetrants are generally known in the art.

For oral administration, the inhibitors of MMP expression and/or activity can be readily formulated by combining the the inhibitors of MMP expression and/or activity with pharmaceutically acceptable carriers well known in the art. Such carriers enable the inhibitors of MMP expression and/or activity to be formulated as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, e.g. lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate.

If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, etc. Additionally, flavoring agents, preservatives, coloring agents and the like can be added.

For buccal administration, the the inhibitors of MMP expression and/or activity can take the form of tablets, lozenges, etc. formulated in conventional manner.

For administration by inhalation, the the inhibitors of MMP expression and/or activity for use according to the present invention are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of gelatin for use in an inhaler or insufflator may be formulated containing a powder mix of the the inhibitors of MMP expression and/or activity and a suitable powder base such as lactose or starch.

The inhibitors of MMP expression and/or activity can also be formulated in rectal or vaginal compositions such as suppositories or retention enemas, e.g, containing conventional suppository bases such as cocoa butter or other glycerides.

In addition to the formulations described previously, the inhibitors of MMP expression and/or activity can also be formulated as a depot preparation. Such long acting formulations may be administered by implantation (for example subcutaneously or intramuscularly) or by intramuscular injection. Thus, for example, the inhibitors of MMP expression and/or activity can be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems can be employed. Liposomes and emulsions are well known examples of delivery vehicles that may be used to deliver the inhibitors of MMP expression and/or activity. Certain organic solvents such as dimethylsulfoxide also may be employed, although usually at the cost of greater toxicity. Additionally, the the inhibitors of MMP expression and/or activity can be delivered using a sustained-release system, such as semipermeable matrices of solid polymers containing the therapeutic agent. Various sustained-release materials have been established and are well known by those skilled in the art. Sustained-release capsules may, depending on their chemical nature, release the the inhibitors of MMP expression and/or activity for a few days, a few weeks, or up to over 100 days. Depending on the chemical nature and the biological stability of the the inhibitors of MMP expression and/or activity additional strategies for stabilization can be employed.

As the inhibitors of MMP expression and/or activity may contain charged side chains or termini, they may be included in any of the above-described formulations as the free acids or bases or as pharmaceutically acceptable salts. Pharmaceutically acceptable salts are those salts which substantially retain the biological activity of the free bases and which are prepared by reaction with inorganic acids. Pharmaceutical salts tend to be more soluble in aqueous and other protic solvents than are the corresponding free base forms.

B) “Genetic” Delivery Methods.

As indicated above, antisense molecules, catalytic RNAs (ribozymes), catalytic DNAs, RNAi, and additionally, knockout constructs, and constructs encoding intrabodies can be delivered and transcribed and/or expressed in target cells (e.g. vascular endothelial cells) using methods of gene therapy. Thus, in certain preferred embodiments, the nucleic acids encoding knockout constructs, intrabodies, antisense molecules, catalytic RNAs or DNAs, etc. are cloned into gene therapy vectors that are competent to transfect cells (such as human or other mammalian cells) in vitro and/or in vivo.

Many approaches for introducing nucleic acids into cells in vivo, ex vivo and in vitro are known. These include lipid or liposome based gene delivery (WO 96/18372; WO 93/24640; Mannino and Gould-Fogerite (1988) BioTechniques 6(7): 682-691; Rose U.S. Pat No. 5,279,833; WO 91/06309; and Felgner et al. (1987) Proc. Natl. Acad. Sci. USA 84: 7413-7414) and replication-defective retroviral vectors harboring a therapeutic polynucleotide sequence as part of the retroviral genome (see, e.g., Miller et al. (1990) Mol. Cell. Biol. 10:4239 (1990); Kolberg (1992) J. NIH Res. 4: 43, and Cornetta et al. (1991) Hum. Gene Ther. 2: 215).

For a review of gene therapy procedures, see, e.g., Anderson, Science (1992) 256: 808-813; Nabel and Felgner (1993) TIBTECH 11: 211-217; Mitani and Caskey (1993) TIBTECH 11: 162-166; Mulligan (1993) Science, 926-932; Dillon (1993) TIBTECH 11: 167-175; Miller (1992) Nature 357: 455-460; Van Brunt (1988) Biotechnology 6(10): 1149-1154; Vigne (1995) Restorative Neurology and Neuroscience 8: 35-36; Kremer and Perricaudet (1995) British Medical Bulletin 51(1) 31-44; Haddada et al. (1995) in Current Topics in Microbiology and Immunology, Doerfler and Böhm (eds) Springer-Verlag, Heidelberg Germany; and Yu et al., (1994) Gene Therapy, 1: 13-26.

Widely used vector systems include, but are not limited to adenovirus, adeno associated virus, and various retroviral expression systems. The use of adenoviral vectors is well known to those of skill and is described in detail, e.g., in WO 96/25507. Particularly preferred adenoviral vectors are described by Wills et al. (1994) Hum. Gene Therap. 5: 1079-1088.

Adeno-associated virus (AAV)-based vectors used to transduce cells with target nucleic acids, e.g., in the in vitro production of nucleic acids and peptides, and in in vivo and ex vivo gene therapy procedures are describe, for example, by West et al. (1987) Virology 160:38-47; Carter et al. (1989) U.S. Pat. No. 4,797,368; Carter et al. WO 93/24641 (1993); Kotin (1994) Human Gene Therapy 5:793-801; Muzyczka (1994) J. Clin. Invst. 94:1351 for an overview of AAV vectors. Lebkowski, U.S. Pat. No. 5,173,414; Tratschin et al. (1985) Mol. Cell. Biol. 5(11):3251-3260; Tratschin, et al. (1984) Mol. Cell. Biol., 4: 2072-2081; Hermonat and Muzyczka (1984) Proc. Natl. Acad. Sci. USA, 81: 6466-6470; McLaughlin et al. (1988) and Samulski et al. (1989) J. Virol., 63:03822-3828. Cell lines that can be transformed by rAAV include those described in Lebkowski et al. (1988) Mol. Cell. Biol., 8:3988-3996.

Widely used retroviral vectors include those based upon murine leukemia virus (MuLV), gibbon ape leukemia virus (GaLV), Simian Immunodeficiency virus (SIV), human immunodeficiency virus (HIV), alphavirus, and combinations thereof (see, e.g., Buchscher et al. (1992) J. Virol. 66(5) 2731-2739; Johann et al. (1992) J. Virol. 66(5):1635-1640 (1992); Sommerfelt et al., (1990) Virol. 176:58-59; Wilson et al. (1989) J. Virol. 63:2374-2378; Miller et al., J. Virol. 65:2220-2224 (1991); Wong-Staal et al., PCT/US94/05700, and Rosenburg and Fauci (1993) in Fundamental Immunology, Third Edition Paul (ed) Raven Press, Ltd., New York and the references therein, and Yu et al. (1994) Gene Therapy, supra; U.S. Pat. No. 6,008,535, and the like). Other suitable viral vectors include, but are not limited to herpes virus, lentivirus, and vaccinia virus.

Alone, or in combination with viral vectors, a number of non-viral vectors are also useful for transfecting cells to express constructs that block or inhibit MMP expression. Suitable non-viral vectors include, but are not limited to, plasmids, cosmids, phagemids, liposomes, water-oil emulsions, polethylene imines, biolistic pellets/beads, and dendrimers.

Liposomes were first described in 1965 as a model of cellular membranes and quickly were applied to the delivery of substances to cells. Liposomes entrap DNA by one of two mechanisms which has resulted in their classification as either cationic liposomes or pH-sensitive liposomes. Cationic liposomes are positively charged liposomes which interact with the negatively charged DNA molecules to form a stable complex. Cationic liposomes typically consist of a positively charged lipid and a co-lipid. Commonly used co-lipids include dioleoyl phosphatidylethanolamine (DOPE) or dioleoyl phosphatidylcholine (DOPC). Co-lipids, also called helper lipids, are in most cases required for stabilization of liposome complex. A variety of positively charged lipid formulations are commercially available and many other are under development. Two of the most frequently cited cationic lipids are lipofectamine and lipofectin. Lipofectin is a commercially available cationic lipid first reported by Phil Felgner in 1987 to deliver genes to cells in culture. Lipofectin is a mixture of N-[1-(2, 3-dioleyloyx) propyl]-N-N-N-trimethyl ammonia chloride (DOTMA) and DOPE.

DNA and lipofectin or lipofectamine interact spontaneously to form complexes that have a 100% loading efficiency. In other words, essentially all of the DNA is complexed with the lipid, provided enough lipid is available. It is assumed that the negative charge of the DNA molecule interacts with the positively charged groups of the DOTMA. The lipid:DNA ratio and overall lipid concentrations used in forming these complexes are extremely important for efficient gene transfer and vary with application. Lipofectin has been used to deliver linear DNA, plasmid DNA, and RNA to a variety of cells in culture. Shortly after its introduction, it was shown that lipofectin could be used to deliver genes in vivo. Following intravenous administration of lipofectin-DNA complexes, both the lung and liver showed marked affinity for uptake of these complexes and transgene expression. Injection of these complexes into other tissues has had varying results and, for the most part, are much less efficient than lipofectin-mediated gene transfer into either the lung or the liver.

PH-sensitive, or negatively-charged liposomes, entrap DNA rather than complex with it. Since both the DNA and the lipid are similarly charged, repulsion rather than complex formation occurs. Yet, some DNA does manage to get entrapped within the aqueous interior of these liposomes. In some cases, these liposomes are destabilized by low pH and hence the term pH-sensitive. To date, cationic liposomes have been much more efficient at gene delivery both in vivo and in vitro than pH-sensitive liposomes. pH-sensitive liposomes have the potential to be much more efficient at in vivo DNA delivery than their cationic counterparts and should be able to do so with reduced toxicity and interference from serum protein.

In another approach dendrimers complexed to the DNA have been used to transfect cells. Such dendrimers include, but are not limited to, “starburst” dendrimers and various dendrimer polycations.

Dendrimer polycations are three dimensional, highly ordered oligomeric and/or polymeric compounds typically formed on a core molecule or designated initiator by reiterative reaction sequences adding the oligomers and/or polymers and providing an outer surface that is positively changed. These dendrimers may be prepared as disclosed in PCT/US83/02052, and U.S. Pat. Nos. 4,507,466, 4,558,120, 4,568,737, 4,587,329, 4,631,337, 4,694,064, 4,713,975, 4,737,550, 4,871,779, 4,857,599.

Typically, the dendrimer polycations comprise a core molecule upon which polymers are added. The polymers may be oligomers or polymers which comprise terminal groups capable of acquiring a positive charge. Suitable core molecules comprise at least two reactive residues which can be utilized for the binding of the core molecule to the oligomers and/or polymers. Examples of the reactive residues are hydroxyl, ester, amino, imino, imido, halide, carboxyl, carboxyhalide maleimide, dithiopyridyl, and sulfhydryl, among others. Preferred core molecules are ammonia, tris-(2-aminoethyl)amine, lysine, ornithine, pentaerythritol and ethylenediamine, among others. Combinations of these residues are also suitable as are other reactive residues.

Oligomers and polymers suitable for the preparation of the dendrimer polycations of the invention are pharmaceutically-acceptable oligomers and/or polymers that are well accepted in the body. Examples of these are polyamidoamines derived from the reaction of an alkyl ester of an α,β-ethylenically unsaturated carboxylic acid or an α,β-ethylenically unsaturated amide and an alkylene polyamine or a polyalkylene polyamine, among others. Preferred are methyl acrylate and ethylenediamine. The polymer is preferably covalently bound to the core molecule.

The terminal groups that may be attached to the oligomers and/or polymers should be capable of acquiring a positive charge. Examples of these are azoles and primary, secondary, tertiary and quaternary aliphatic and aromatic amines and azoles, which may be substituted with S or O, guanidinium, and combinations thereof. The terminal cationic groups are preferably attached in a covalent manner to the oligomers and/or polymers. Preferred terminal cationic groups are amines and guanidinium. However, others may also be utilized. The terminal cationic groups may be present in a proportion of about 10 to 100% of all terminal groups of the oligomer and/or polymer, and more preferably about 50 to 100%.

The dendrimer polycation may also comprise 0 to about 90% terminal reactive residues other than the cationic groups. Suitable terminal reactive residues other than the terminal cationic groups are hydroxyl, cyano, carboxyl, sulfhydryl, amide and thioether, among others, and combinations thereof. However others may also be utilized.

The dendrimer polycation is generally and preferably non-covalently associated with the polynucleotide. This permits an easy disassociation or disassembling of the composition once it is delivered into the cell. Typical dendrimer polycation suitable for use herein have a molecular weight ranging from about 2,000 to 1,000,000 Da, and more preferably about 5,000 to 500,000 Da. However, other molecule weights are also suitable. Preferred dendrimer polycations have a hydrodynamic radius of about 11 to 60 Å., and more preferably about 15 to 55 Å. Other sizes, however, are also suitable. Methods for the preparation and use of dendrimers in gene therapy are well known to those of skill in the art and describe in detail, for example, in U.S. Pat. No. 5,661,025.

Where appropriate, two or more types of vectors can be used together. For example, a plasmid vector may be used in conjunction with liposomes. In the case of non-viral vectors, nucleic acid may be incorporated into the non-viral vectors by any suitable means known in the art. For plasmids, this typically involves ligating the construct into a suitable restriction site. For vectors such as liposomes, water-oil emulsions, polyethylene amines and dendrimers, the vector and construct may be associated by mixing under suitable conditions known in the art.

C) Effective Dosages.

The inhibitors of MMP expression and/or activity will generally be used in an amount effective to achieve the intended purpose (e.g. to reduce or prevent secondary neurological damage following trauma, or improve recovery from neurological damage). In preferred embodiments, the MMPI(s) utilized in the methods of this invention are administered at a dose that is effective to partially or fully inhibit the hydrolytic activity of one or more matrix metalloproteinase enzymes (e.g. MMP-9) (e.g., a statistically significant decrease at the 90%, more preferably at the 95%, and most preferably at the at the 98% or 99% confidence level). Preferred effective amounts are those that reduce or prevent secondary neurological damage or improve recovery from neurological damage. The compounds can also be used prophalactically at the same dose levels.

Typically, the inhibitors of MMP expression and/or activity, or pharmaceutical compositions thereof, are administered or applied in a therapeutically effective amount. A therapeutically effective amount is an amount effective to reduce or prevent secondary neurological damage or improve recovery from neurological damage. Determination of a therapeutically effective amount is well within the capabilities of those skilled in the art, especially in light of the detailed disclosure provided herein.

For systemic administration, a therapeutically effective dose can be estimated initially from in vitro assays. For example, a dose can be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art. One skilled in the art could readily optimize administration to humans based on animal data.

Dosage amount and interval may be adjusted individually to provide plasma levels of the inhibitors which are sufficient to maintain therapeutic effect.

Dosages for typical therapeutics, particularly for MMPIs, are known to those of skill in the art. Moreover, such dosages are typically advisorial in nature and may be adjusted depending on the particular therapeutic context, patient tolerance, etc. Single or multiple administrations of the compositions may be administered depending on the dosage and frequency as required and tolerated by the patient.

In certain embodiments, an initial dosage of about 1μ, preferably from about 1 mg to about 1000 mg per kilogram daily will be effective. A daily dose range of about 5 to about 75 mg is preferred. The dosages, however, may be varied depending upon the requirements of the patient, the severity of the condition being treated, and the compound being employed. Determination of the proper dosage for a particular situation is within the skill of the art. Generally, treatment is initiated with smaller dosages which are less than the optimum dose of the compound. Thereafter, the dosage is increased by small increments until the optimum effect under the circumstance is reached. F or convenience, the total daily dosage may be divided and administered in portions during the day if desired. Typical dosages will be from about 0.1 to about 500 mg/kg, and ideally about 25 to about 250 mg/kg.

In cases of local administration or selective uptake, the effective local concentration of the inhibitors may not be related to plasma concentration. One skilled in the art will be able to optimize therapeutically effective local dosages without undue experimentation. The amount of inhibitor administered will, of course, be dependent on the subject being treated, on the subject's weight, the severity of the affliction, the manner of administration and the judgment of the prescribing physician.

The therapy may be repeated intermittently. The therapy may be provided alone or in combination with other drugs and/or procedures.

D) Toxicity.

Preferably, a therapeutically effective dose of the inhibitors of MMP expression and/or activity described herein will provide therapeutic benefit without causing substantial toxicity.

Toxicity of the inhibitors described herein can be determined by standard pharmaceutical procedures in cell cultures or experimental animals, e.g., by determining the LD₅₀ (the dose lethal to 50% of the population) or the LD₁₀₀ (the dose lethal to 100% of the population). It is noted that toxicity of numerous MMPIs is well characterized. The dose ratio between toxic and therapeutic effect is the therapeutic index. Inhibitors which exhibit high therapeutic indices are preferred. The data obtained from these cell culture assays and animal studies can be used in formulating a dosage range that is not toxic for use in human. The dosage of the inhibitors described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch.1, p.1).

IV. Kits.

In another embodiment, this invention provides therapeutic kits for practice of the methods of this invention. Such kits preferably include a container containing one or more matrix metalloproteinase inhibitors (MMPIs). The MMPIs can be formulated in combination with a pharmaceutically acceptable excipient and/or in a unit dosage form.

The kit can comprise packaging that retains and presents the medicants (MMPIs) at separate respective consecutive locations identified by visibly discernible indicia and the times at which the medicants are to be taken by the patient. In various embodiments, the times can include each day of the week and specified times within each day presented in the form of a chart located on one face of the package wherein the days of the week are presented and the times within each day the medicants are to be taken are presented in systematic fashion.

In addition, the kits can include instructional materials containing directions teaching the use of one or more MMPIs to reduce neurological damage following trauma to nervous tissue in a mammal, and/or to reducing abnormal vascular permeability associated with spinal cord injury, and/or to improve recovery of neurological function following injury to neurological tissue. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

Matrix Metalloproteinases Limit Functional Recovery After Spinal Cord Injury by Modulation of Early Vascular Events

Inflammation in general and proteinases generated as a result are likely mediators of early secondary pathogenesis after spinal cord injury. We report that matrix metalloproteinase-9 (MMP-9) plays an important role in blood-spinal cord barrier dysfunction, inflammation, and locomotor recovery. MMP-9 was present in the meninges and neurons of the uninjured cord. MMP-9 increased rapidly after a moderate contusion spinal cord injury, reaching a maximum at 24 hours, becoming markedly reduced by 72 hours, and not detectable at 7 days post injury. It was seen in glia, macrophages, neutrophils and vascular elements in the injured spinal cord at 24 hours post injury. The natural tissue inhibitors of MMPS were unchanged over this time course. MMP-9 null mice exhibited significantly less disruption of the blood-spinal cord barrier, attenuation of neutrophil infiltration, and significant locomotor recovery as compared to the wild type. Similar findings were observed in mice treated with a hydroxamic acid MMP inhibitor from 3 hours to 3 days post injury, as compared to the vehicle controls. Moreover, the area of residual white matter at the lesion epicenter was significantly greater in the inhibitortreated group. This study provides evidence that MMP-9 plays a key role in abnormal vascular permeability and inflammation within the first 3 days after spinal cord injury and that blockade of MMPs during this critical period attenuates these vascular events and leads to improved locomotor recovery. Our findings suggest that early inhibition of MMPs may be an efficacious strategy for the spinal cord injured patient.

Introduction.

Each year there are approximately 10,000 spinal cord injuries that result in permanent disabilities. There is considerable evidence that functional recovery after spinal cord injury is not simply the consequences of the initial mechanical destruction of tissue but is also attributed to the evolution of complex secondary events that contribute to early as well as delayed cell injury. Proteinases, and in particular matrix metalloproteinases (MMPs), are likely mediators of early secondary vascular pathogenesis after spinal cord injury.

MMPs are a family of extracellular zinc and calcium dependent endopeptidases (Birkedal-Hansen et al. (1993) Crit. Rev. Oral Biol. Med. 4:197-250) that degrade the extracellular matrix and other extracellular proteins (Sternlicht (1999) P. 503-603 In: Extracellular Matrix Proteinases. In: Guidebook to the Extracellular Matrix, Anchor and Adhesion Proteins (Kreis T, Vale R, eds), pp Oxford University Press, N.Y.; Sternlicht (2001) Annu Rev Cell Dev Biol., 17:463-516). MMPs are essential for remodeling of the extracellular matrix, tissue morphogenesis, and wound healing (Werb, 1997). However, excessive proteolytic activity of MMPs can be detrimental, leading to numerous pathologic conditions including disruption of the blood-brain barrier (Rosenberg et al. (1994) Laboratory Investigation 71:417-422; Rosenberg et al. (1995) Brain Res., 703:151-155; Rosenberg and Navratil (1997) Neurology 48:921-926; Mun-Bryce and Rosenberg (1998) Am. J. Phpysiol., 274:R1203-1211; Rosenberg et al. (1998) Stroke 29:2189-2195; Yong et al. (2001) Nat Rev Neurosci 2:502-511; Mun-Bryce and Rosenberg (1998) Am. J. Phpysiol., 274:R1203-1211). MMP-9 degrades gelatin (denatured collagens), collagen IV, V, XI, elastin, vitronectin, myelin basic protein and other substrates (Vu and Werb (1998) Gelatinase B: structure, regulation, and function. San Diego: Academic Press). This protease is predominantly expressed by inflammatory cells including macrophages, lymphocytes, and neutrophils as well as endothelial cells (Mainardi et al. (1984) Collagen and Related Research 4:479-492; Hibbs et al. (1987) J. Clin. Invest., 80:1644-1650; Murphy et al. (1989) Biolchem. J., 258:463-472; Wilhelm et al. (1989) [published erratum appears in J Biol Chem 1990 December; 265(36):22570]. J. Biol, Chem., 264:17213-17221). Recent studies suggest that MMP-9 inactivates alpha 1-antitrypsin, the primary physiologic inhibitor of leukocyte elastase, a step that is central to leukocyte migration (Liu et al. (1998) J. Exp. Med., 188:475-482). In the central nervous system, there is a low constitutive expression of MMP-9 in microglia, astrocytes and hippocampal neurons, and it can be induced in astrocytes, microglia/macrophages and hippocampal cells (Backstrom et al. (1996) J. Neurosci., 16:7910-7919; Cuzner et al. (1996) J. Neuropathology Exp. Neurol., 55:1194-1204; Gottschall and Deb (1996) Neuroimmunomodulation 3:69-75; Liu et al. (1998) J. Exp. Med., 188:475-482; Yong et al. (2001) Nat Rev Neurosci 2:502-511).

We have focused on the role of MMP-9 because of its established link to disruption of the blood-brain barrier, inflammation, and tissue injury. MMP-9 has been implicated in abnormal vascular permeability (Rosenberg et al. (1994) Laboratory Investigation 71:417-422; Rosenberg et al. (1995) Brain Res., 703:151-155; Mun-Bryce and Rosenberg (1998) Am. J. Phpysiol., 274:R1203-1211), associated with either hemorrhagic injury (Rosenberg et al. (1994) supra.) or inflammation (Mun-Bryce and Rosenberg (1998) supra.). Thus, abnormal increases in MMP-9 in both inflammatory cells as well as endothelial cells may collectively impair barrier function by degrading the vascular basement membrane. There is also evidence that MMP-9 increases in ischemic brain injury (Rosenberg et al. (1996) Neurology 46:1626-1632; Romanic et al. (1998) Stroke 29:1020-1030) and that the administration of a monoclonal antibody to MMP-9 reduces the hemispheric infarct size (Id.). Most recently, it has been shown that methylprednisolone, the only therapeutic agent approved by the Food and Drug Administration, suppresses the expression of MMP-9 after spinal cord injury (Xu et al. (2001) J. Neurosci., 21:92-97).

Materials and Methods.

Generation of the Experimental Models.

Surgical Procedures.

All procedures were performed according to protocols approved by the University of California Committee on Research (San Francisco, Calif.). MMP-9 null and wild type littermates were generated as previously described (Vu et al., 1998) and bred on an FVBn background. The wild type mice were obtained from the negative littermates of the backcrosses into the FVBn background. The MMP-9 null mouse has a mild developmental delay in bone formation (Vu et al. (1998) Cell 93:411-422). However, by six weeks of age these animals have an axial skeleton indistinguishable from the wild type mouse. These mice have a normal lifespan and there are no phenotypic differences between the MMP-9 null and wild type mice. All studies described below were conducted in a blinded fashion.

Adult, male mice (4-6 months of age), were anesthetized with 2.5% Avertin (0.02 ml/g bw, i.p.) and maintained at 37° C. throughout the experiment by using a warming blanket placed under the animal. A contusive injury was performed based upon modifications of procedures originally described by Kuhn and Wrathall (1998) J. Neurotrauma 15:125-140. Briefly, using aseptic techniques the spinous process and laminae of T8 were removed and a circular region of dura, approximately 2.4 mm in diameter, was exposed. After stabilization of the vertebral column, a 2 gm weight was dropped 5.0 cm onto the exposed dura. After injury, the overlying skin was closed with wound clips. Postoperative care included manual expression of each animal's bladder until recovery of reflex emptying.

Inhibitor Studies.

Wild type mice were subjected to spinal cord injury as described in the previous section. All mice were treated with either GM 6001 (AMS Scientific, Inc., Concord, Calif. 100 mg/kg in 4% methylcellulose, i.p.), a general inhibitor of MMPs, or vehicle (4% methylcellulose, i.p.), at 3 hours post injury. Animals were treated every 12 hours (100 mg/kg in 4% methylcellulose, i.p.), for the first 3 days post injury.

Zymography

Gelatin Zymography.

Samples of spinal cord, prepared from the epicenter, were quick-frozen at −80° C. Each sample was weighed and homogenized in (1:4 weight to volume) in lysis buffer containing 50 mM Tris-HCl (pH 8.0), 150 mM NaCl, 1% NP-40, 0.5% deoxycholate, 0.1% SDS. Soluble and insoluble extracts were separated by centrifugation and stored at −20° C. Equal amounts of the supernatant were analyzed by gel zymography as previously described (Herron et al., 1986) on 10% SDS-polyacrylamide gels, copolymerized with substrate (1 mg/ml gelatin). The proteins were renatured by incubation in 2.5% Triton-X100, and then incubated in substrate buffer (50 mM Tris-HCl, pH 8.5 mM CaCl₂) for 24 to 36 hours at 37° C. to enable the MMP-9 and other gelatinases to cleave the gelatin. After rinsing in water, each gel was stained with Coomassie Blue for 4 hours and destained in 50% methanol. Negative staining is indicative of the location of active protease bands. After exposure to SDS during gel separation, proenzymes, present in tissue extracts, are activated without proteolytic cleavage. To inhibit MMP proteolytic activities, substrate gels were incubated in substrate buffer with 4 mM 1,10-phenanthroline (Sigma, St. Louis, Mo.), as described (Adler et al. 1990) Development 110:211-220). This control ensured that the measured activity corresponded to matrix metalloproteinase activity. The identities of MMPs were based on their molecular weights.

Reverse Zymography.

Reverse zymography was used to identify physiologic inhibitors (TIMP-1 and TIMP-2). The gel was prepared as described in the previous paragraph with the exception that gelatinases were also added to the SDS-gelatin gel. The gelatinases degrade the gel except in those regions in which there is inhibitory activity. As a result, TIMP-1 activity is visualized in Coomassie blue stained and destained gels as dark blue bands.

In Situ Zymography.

In situ zymography was used to detect and localize enzyme activity in tissue sections. The uninjured and injured (24 hours post injury) spinal cords were quickly removed without fixation and frozen at −80° C. Sections (16 μm) were cut on a cryostat and were incubated in 0.05 M Tris-HCl, 0.15 M NaCl, 5 mM CaCl₂, 0.2 mM NaN₃, pH 7.6, containing 40 μg FITC-labeled DQ gelatin (Molecular Probes), at 37° C. for 1 hour. The gelatin has a fluorescent tag remains caged (does not fluoresce) until the gelatin is cleaved by gelatinase activity, such as MMP-9 (or MMP-2). This method detects regionally specific gelatinolytic activity, but does not distinguish between gelatinases. Reaction product was visualized by fluorescence microscopy.

Immunocytochemistry and Histochemistry

Immunocytochemistry.

At a designated time point, animals were deeply anesthestized and perfused with 4% paraformaldehyde in 0.1 M phosphate buffer saline (PBS), pH 7 4. The spinal cord was removed, rinsed in PBS and either prepared for embedding in paraffin or cryoprotected in sucrose (20% in PBS), and frozen. For paraffin embedding, tissue was dehydrated through graded alcohols and xylene, and embedded in paraffin. Sections, 5-10 μm in thickness, were cut using a Leica 2135 microtome and deparaffinized. Frozen sections (10-15 μm) were cut on a cryostat.

Conventional immunocytochemistry was performed on either deparaffinized sections or cryostat sections. Dilutions used for rabbit anti-mouse MMP-9 (Behrendtsen et al. (1992) Development 114:447-456) was 1/200, 1/500-1000 for porcine antimouse glial fibrillary acidic protein (Sigma, St. Louis, Mo.), 1:200 for rabbit anti-mouse PECAM-1 (BDPharMingen Int. , San Diego, Calif.), and 1:5 for the macrophage-specific rat anti-mouse F4/80 (Austyn and Gordon (1981) Eur. J. Immunol., 11:805-815). These antibodies were prepared in blocking solution consisting of 1% sheep serum and unless other specified, blocking agents and secondary antibodies provided by the Tyramide Signal Amplification (TSA) Direct and Indirect Kits (Molecular Probes, Eugene, Oreg.), following manufacturer's instructions. Incubation of primary antibodies was overnight at 4° C., with the exception of anti-GFAP which was for 30 minutes at room temperature. Secondary antibodies were used at a 1:500 dilution. Final detection of the signal employed either a fluorescent or biotinylated tyramide derivative and visualized using a peroxidase substrate. The anti-GFAP was conjugated to Cy3 (Sigma) and directly visualized. Immunocytochemical controls consisted of omission of the primary antibody. All images were digitally captured on a Leica microscope, equipped with a CCD camera (SPOT, model 1. 3. 0, Diagnostic Instruments, Inc.), and imaged using Photoshop 6.0™ (Adobe System, San Jose Calif.).

Histochemistry.

Neutrophils were identified in the injured cord at 42 days post injury by means of a choroacetate esterase stain (naphthol AS-D chloroacete esterase Kit, Sigma). The protocol was as described by the manufacturer with several exceptions. All cords were fixed by intravascular perfusion with 4% paraformaldehyde. These fixed sections were then incubated for 20 minutes in the esterase stain. Serial sections, 14 μm in thickness, were obtained from a 1.0 cm length of cord, centered over the region of maximal damage. Every 4^{th} section was mounted on slides for evaluation. One section, exhibiting maximal neutrophil infiltration, was selected from a 1.0 mm length of cord, centered over the impact site. In addition, 1 section, 0.8 mm rostral and 1 section, 0.8 mm caudal to the lesion epicenter, were selected. The number of neutrophils within each of these sections was determined from digital images, captured with a CCD camera, as described in the preceding paragraph at 40×magnification. Contrast for each image was optimized using Adobe Photoshop 6.0™. Only darkly stained structures, greater than 38 μm² (7 μm in diameter) were counted. For the rostral and caudal sections, the numbers of neutrophils within the entire cross sections were determined and summed. For the epicenter, neutrophils were quantified within specific regions of the cross section. Those neutrophils were counted if they resided in rectangular boxes, centrally positioned in the right and left dorsal horns (231×126 μm²), dorsal columns (173×96 μm²), right and left lateral white matter (2211×384 μm²), pericentral grey matter (369×173 μm²), and right and left ventral white matter (373×164 μm²). Values were expressed at a ratio of the summed number of neutrophils in each of the rostral and caudal segments divided by the number of neutrophils at the epicenter.

The area of residual white matter at the epicenter was determined in vehicle-and drug-treated wild type mice at 42 days post injury. We selected residual white matter for analysis because we had previously shown that it is the best single measurement for characterizing the degree of injury in the contused spinal cord and is predictive of motor recovery (Noble and Wrathall (1985) Exp. Neurol., 88:135-149). Serial cross sections from the lesion epicenter, prepared from animals that had been euthanized at 42 days post injury, were stained for myelin using Luxol fast blue. Stained cross sections, prepared from a 5 mm segment, centered over the impact site, were selected for analysis. The section that exhibited the greatest loss of white matter, as demonstrated by Luxol Fast Blue, was selected for subsequent analysis. These sections were captured with a Spotcam camera mounted on a Nikon Optiphot and analyzed using Photoshop 6.0™. In each captured image, a histogram was generated that resulted in 2 distinct peaks, corresponding to areas of Luxol fast blue staining and background staining. Each image was then adjusted such that only pixels related to Luxol fast blue were visible. These pixels were quantified and expressed per unit area.

Barrier Permeability Studies

Permeability to Horseradish Peroxidase (HRP).

Barrier permeability to HRP was evaluated in spinal cord injured wild type and MMP-9 null mice and mice treated with either GM6001 or vehicle. Mice were anesthetized and administered HRP (Type II, 75 mg/kg in 0.4 ml 0.9% saline, i.p.) 10 minutes prior to euthanasia at 24 hours post injury. The mice were perfused with fixative, as described for the immunocytochemistry. After removal of the spinal cord the tissue was rinsed in buffer, cryoprotected, and frozen. A 1.0-cm length of cord, centered over the impact site, was selected for serial sectioning. Cross sections, 20 μm in thickness, were cut using a cryostat. Sections were dehydrated in graded alcohols, cleared, and mounted on slides. Reaction product was visualized using 3,3-diaminobenzidine tetrachloride (DAB) as the chromogen. Every 50^{th} section was selected for semiquantitative analysis. Each cross section was subdivided into 11 regions, corresponding to the dorsal columns, pericentral grey matter and ventral white matter and right and left regions of each of the following: pericentral, lateral white matter, peripheral lateral white matter, dorsal horns, and ventral horns. The extent of extravasation was evaluated on a 3 point, graded scale as follows: 1=light staining that is limited to discrete patches, 2=light staining throughout the region and/or darkly stained patches, 3=darkly stained throughout the region. A maximal score for any given section was 33.

Permeability to Luciferase.

MMP-9 null and wild type mice and mice treated with either vehicle or GM 6001 were re-anesthetized at 24 hours post injury and injected i. v. with a 1:1 solution consisting of recombinant luciferase (“Quantilum”, 1 mg/ml in Luciferase Storage Buffer, Promega, Madison, Wis.) and PBS/BSA (2.7 mM potassium chloride, 1.5 mM potassium phosphate, 8.1 mM sodium phosphate, 0.8% sodium chloride, 0.001% bovine serum albumin). Animals were then placed on a warming blanket to maintain body temperature.

Each animal was euthanized, 30 min after injection of luciferase, and the spinal cord was quickly removed. A 3-mm length of cord, centered over the impact site, was homogenized in a 1:50 dilution by weight of 1X cell lysis buffer (25 mM Tris, pH 7. 8, 2 mM trans 1,2 diamino cyclohexane n,n,n′,n′ tetraacetate monohydrate, 2 mM dithiothreitol, 10% glycerol, 1% Triton X-100). Lysates were centrifuged (12,000 rpm for 8 minutes) to remove cellular debris, and the supernatants were brought to a final dilution of 1:5000. Enzyme activities, measured in 10 μl aliquots of the dilution, were based on luminescence using a luciferase assay kit (Promega) and a luminometer (TD-20/20, Turner Designs, Sunnyvale, Calif.) with a 10 second measuring time. Values were expressed as the ratio of activity within the injured segment relative to an internal control (cervical segment).

Functional Assessment

Locomotor recovery was assessed using an open field testing paradigm, the BBB Locomotor Rating Scale, that is based upon a 21 point scale originally developed in the spinal cord injured rat (Basso et al. 1995) J. Neurotrauma 12:1-21). This scale assesses 10 distinct categories that range from limb movement to tail position and involve detailed observations of joint movement, stepping, and coordination. Uninjured animals exhibit a locomotor score of “21” whereas animals that exhibit complete hind limb paralysis are scored as a “0”. Animals that are moderately injured typically show recovery over time and exhibit a locomotor score of between 10 and 11 by about 6 weeks post injury (Basso et al. 1995) J. Neurotrauma 12:1-21; Basso et al. (1996) J. Neurotrauma, 13:343-359). Spinal cord injured animals were tested on days 1 and 3 post injury and weekly thereafter for 6 weeks. Each animal was tested within an enclosed arena of clear acrylic (53 cm×108 cm×5.5 cm) that was supported over a mirror. Positioning of the limbs and locomotion was then observed by either directly or indirectly (via the mirror) viewing the animal.

Quantitative Analysis

The mean values for luciferase permeability, neutrophil infiltration, locomotor recovery, and residual white matter for each control and experimental group are reported ± standard deviations. MMP-9 nulls and drug treated groups were compared with their respective controls (wild types and vehicle treated mice) using unpaired Students's t tests. Statistical significance was defined at P<0.05.

Results.

MMP-9 Activity Increases After Spinal Cord Injury

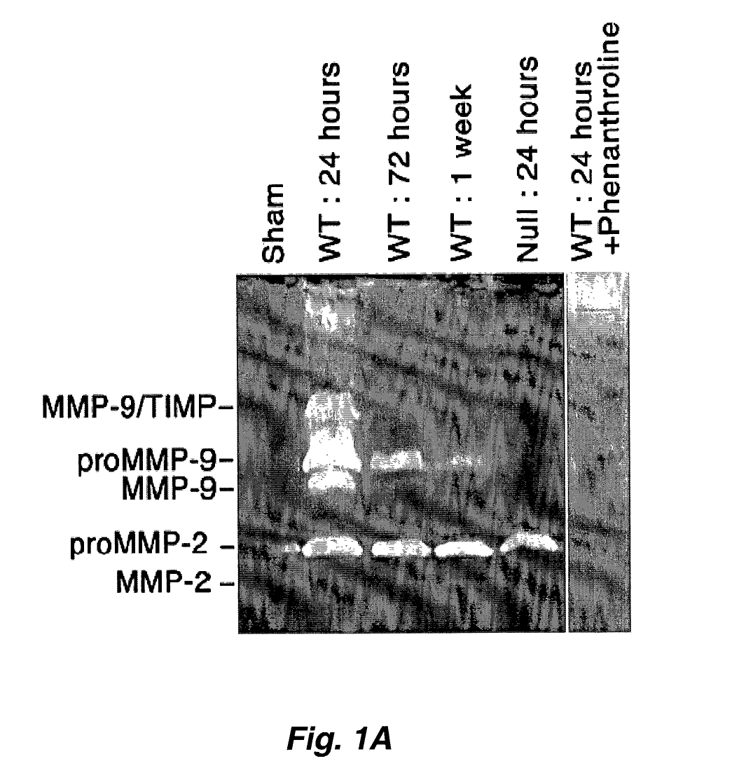

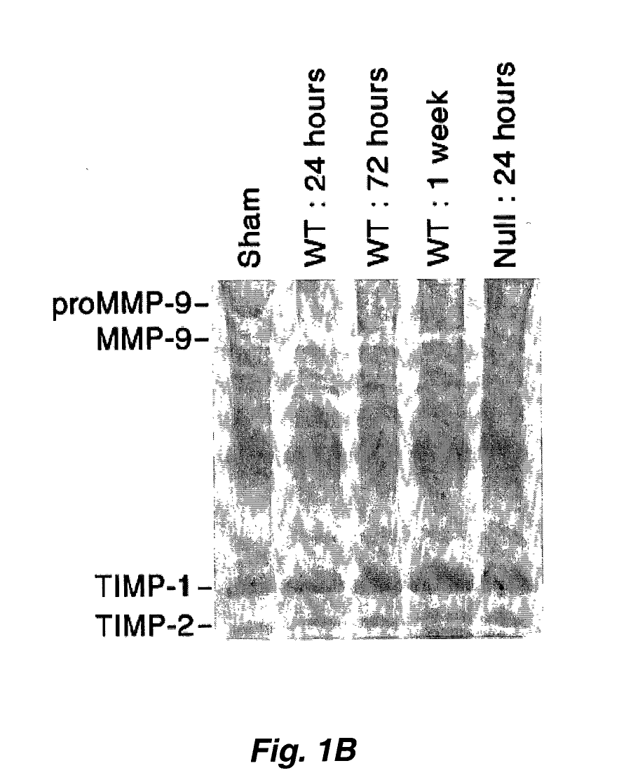

We first analyzed MMP activity in the spinal cord in wild-type mice after injury by gelatin zymography (FIG. 1A). Animals were subjected to contusive spinal cord injury and euthanized at 24 h, 72 h, and 1 week following injury. Samples were also taken from uninjured (laminectomized) wild type mice and MMP-9 null mice 24 h following injury. In all animals, a 4 mm segment of cord corresponding to the center of the injury was homogenized and subject to gelatin zymography. In the uninjured mouse, no gelatinase activity was detected. In the wild type injured mouse, MMP-9 activity was strongest at 24 h post injury, with bands corresponding to the MMP-9 active form, the inactive zymogen, and MMP-9 /TIMP-1 complexes. This activity was reduced by 72 h, and only the inactive zymogen was present by one week post injury. The inactive form of MMP-2 (gelatinase A) appeared in all injured samples. The MMP-9 null mouse did not have the MMP-9 bands, as would be expected, but did not show a compensatory increase in MMP-2. A general inhibitor of metalloproteinases, 1,10 phenanthroline, completely blocked the gelatinolytic activity, thus confirming that these bands were due to metalloproteinases (FIG. 1A).

MMP-9 is Present in Uninjured Meninges and Motoneurons and in Blood Vessels, Macrophages and Astrocytes in Injured Spinal Cord

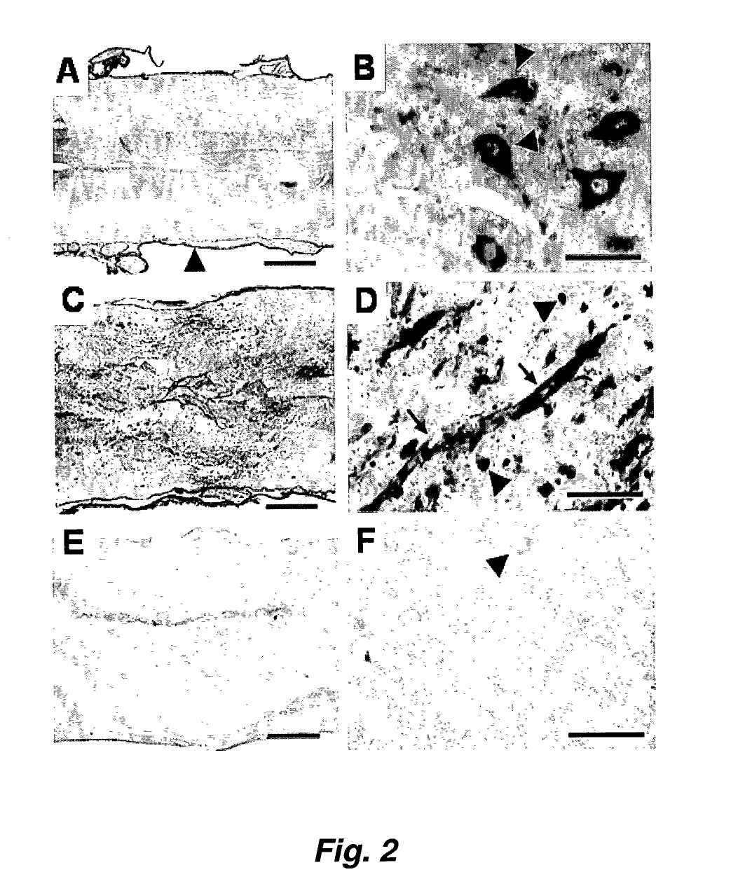

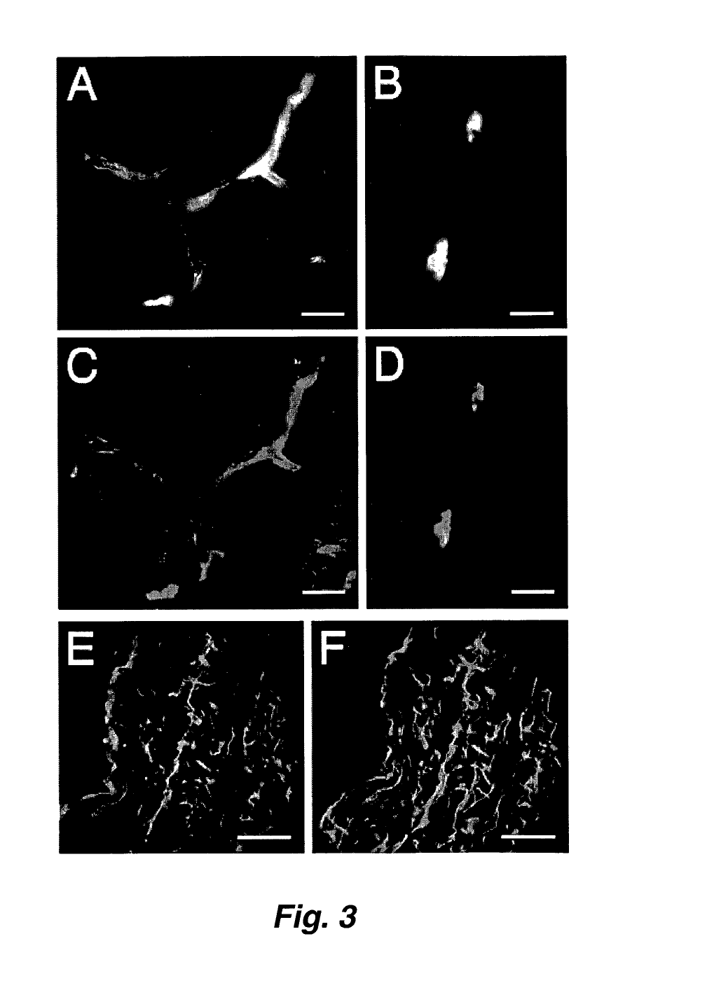

MMP-9 was localized in the meninges and in ventral horn motoneurons in the uninjured spinal cord of wild type mice (FIG. 2). There was no evidence for expression of MMP-9 in other neurons or in glia. At 24 hours post injury, there was a similar pattern of expression of MMP-9 in segments of spinal cord that was at least one segment removed from the injury (FIG. 2). However, at the epicenter (the region of maximal damage) and the immediately adjacent tissue, there was pronounced induction of MMP-9 that was associated with blood vessels as well as cells that were identified as macrophages, and astrocytes by double immunolabeling (FIGS. 2, 3).

In situ Zymography of Gelatinolytic Activity Shows Increased Activity After Injury

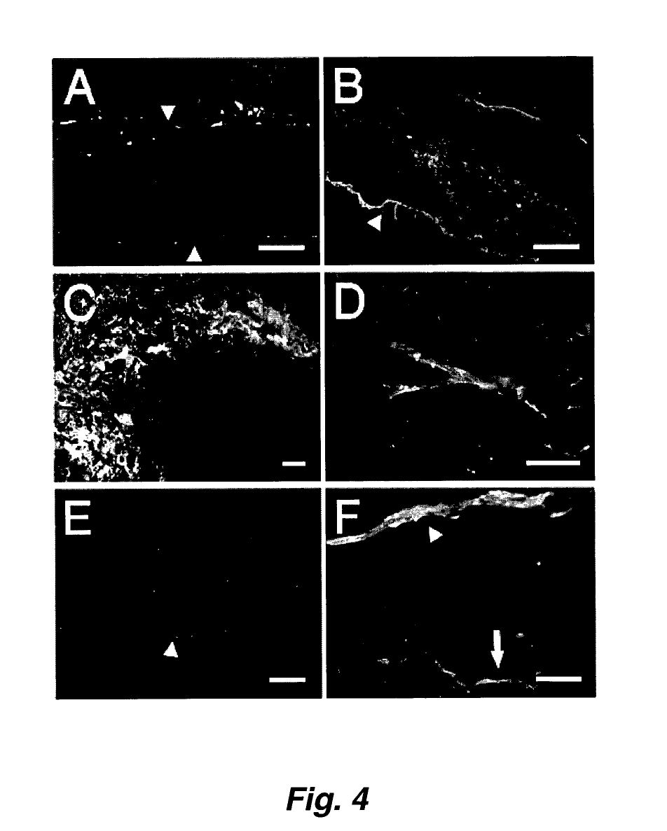

By in situ zymography on sections of uninjured spinal cord in the wild type animals, gelatinolytic activity was primarily restricted to the meninges (FIG. 4). The traumatized spinal cord at 24 hours post contusion injury exhibited several distinct changes in gelatinolytic activity. There was increased activity associated within the meninges bordering the impact site and abundant activity within the epicenter (FIG. 4). Gelatinase activity was associated with a variety of cell areas including blood vessels (FIG. 4). It is noteworthy that gelatinase activity was detected in the spinal cord of the MMP-9 null animal after injury (FIG. 4). This is not surprising since it is known that other members of the MMP family can contribute to gelatinolytic activity (Yong et al. (2001) Nat Rev Neurosci 2:502-511).

Lack of MMPs Blunts the Blood-spinal Cord Barrier Breakdown After Spinal Cord Injury.

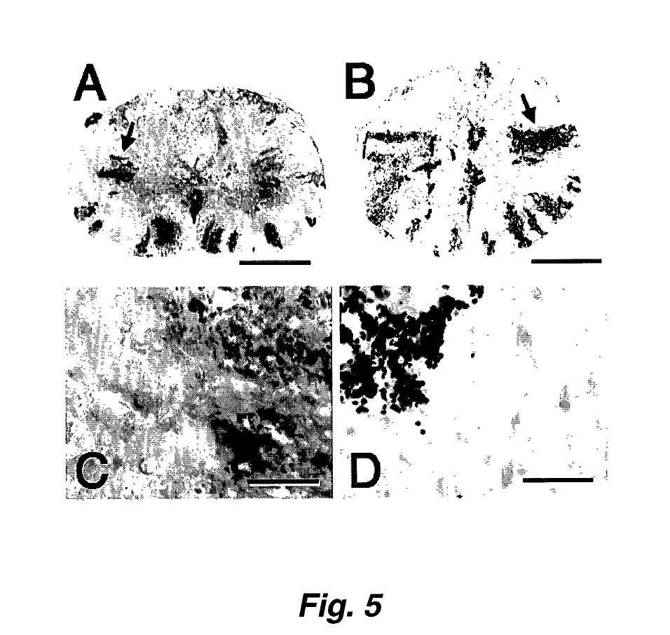

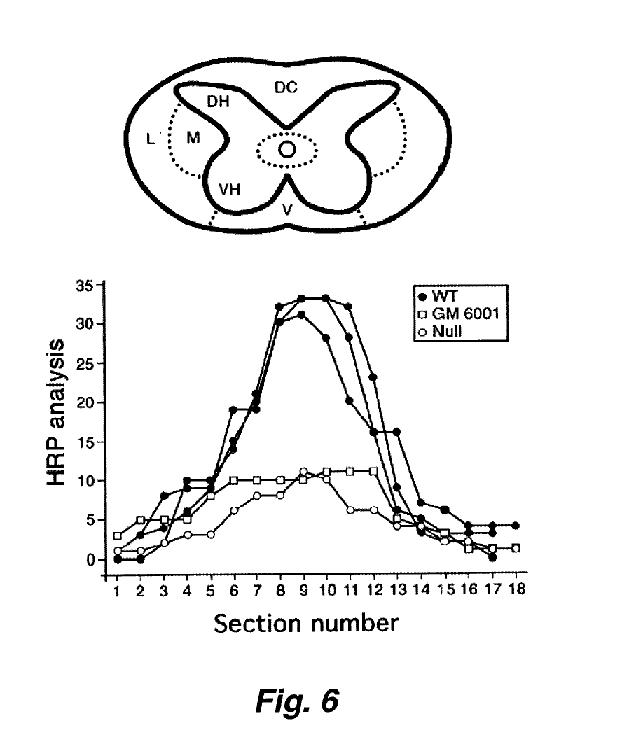

We have previously shown that spinal cord injury results in prominent breakdown of the blood-spinal cord barrier to endogenous proteins as well as to HRP (Noble and Wrathall (1989) Brain Res., 482:57-66). To determine whether increased MMP-9 activity after spinal cord injury contributes to this abnormal permeability we performed two types of experiment. In the first experiment, we compared the blood-spinal cord barrier to HRP 24 hours after spinal cord injury in wild type mice with MMP-9 null mice and wild type mice that were treated with the MMP inhibitor GM 6001. The lesion epicenter in the wild type and null mice were characterized by prominent intraparenchymal hemorrhage (FIG. 5). The magnitude of hemorrhage in animals was quite variable. This finding is consistent with other studies which have demonstrated that the extent of intraparenchymal hemorrhage does not correlate with injury severity after graded spinal cord contusion injuries nor is it a good predictor of functional outcome (Noble and Wrathall (1989) Exp. Neurol., 103:34-40). Red blood cells appeared as dark brown, due to the peroxidatic-like activity of hemoglobin, and were distributed in a “spoke-like” pattern that radiated outward from the center of the cord. Importantly, there was a distinct difference in the pattern of barrier permeability to HRP in the wild type as compared to the MMP-9 null mice and mice treated with GM 6001 (FIGS. 5, 6). HRP, which appeared as a diffuse, light brown reaction product, was maximally expressed in the lesion epicenter of the wild type. In contrast, there were only light patches of HRP reaction product in the epicenter of the MMP-9 null and drug-treated animals (FIG. 5). Although barrier permeability was most dramatic at the lesion epicenter in all animals, segments rostral and caudal to the lesion of the wild type mice also exhibited abnormal permeability. In contrast, this axial distribution of abnormal barrier permeability was not observed in the MMP-9 null or GM 6001-treated animals (FIG. 6).

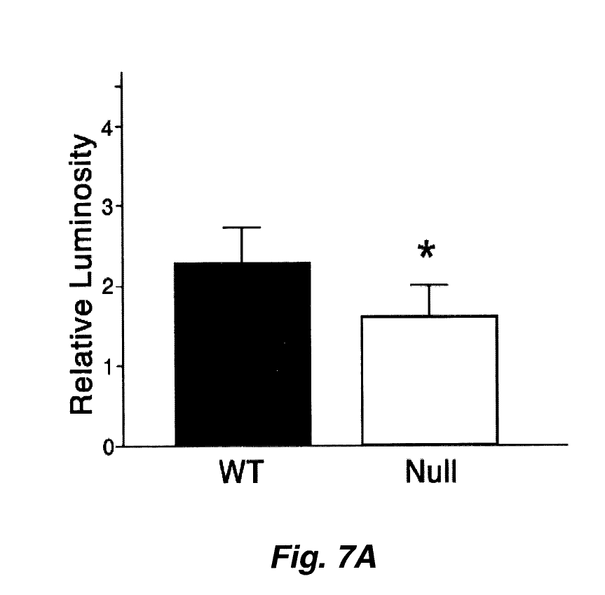

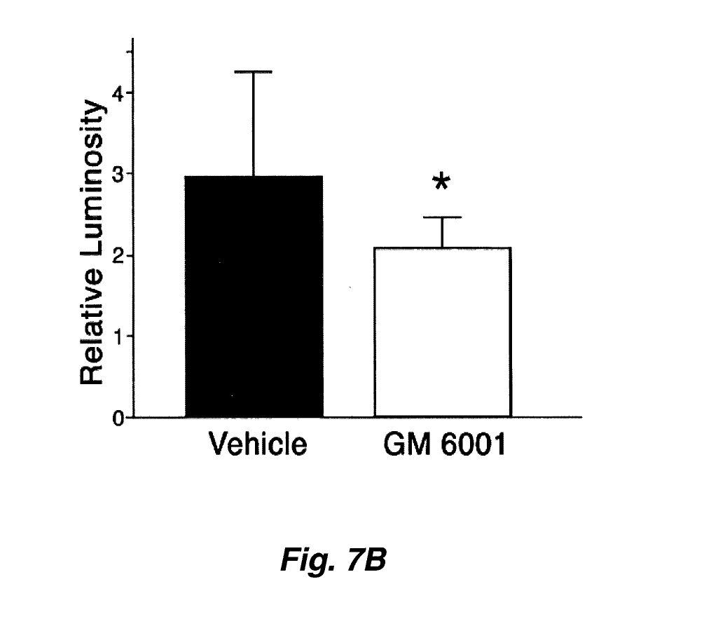

In the second type of experiment, barrier breakdown to the protein luciferase was quantified within the homogenates prepared from the lesion epicenter of spinal cord injured MMP-9 null and wild type mice and wild type mice that were treated with either vehicle or GM 6001 (FIG. 7). Similar to the anatomical studies described in the HRP study, abnormal barrier permeability was significantly attenuated in the MMP-9 null mice as compared to the wild types and in the drug treated as compared to the vehicle treated animals.

Blocking MMPs Decreases Neutrophil Infiltration

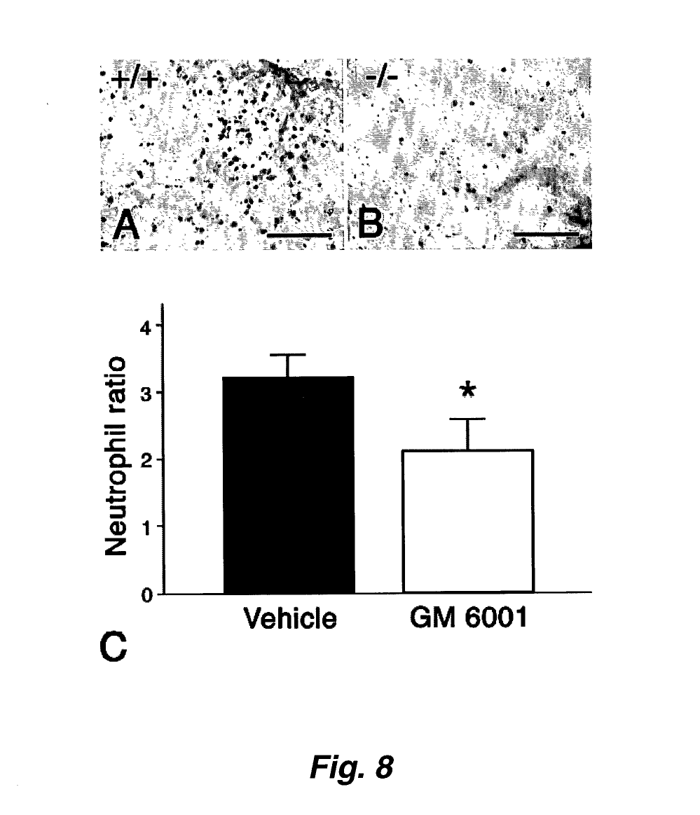

Acute inflammation is a normal response to injury. We found that 70%-74% of all neutrophils within the lesion epicenter of the vehicle and drug treated animals resided in the white matter. Moreover, we observed that there were fewer neutrophils infiltrating within the lesion epicenter of MMP-9 null as compared to the wild type mice at 24 hours post injury (FIG. 8). When neutrophil infiltration was quantified in spinal cord injured mice that were treated with either vehicle or the MMP inhibitor GM 6001 (FIG. 8), we observed a significant reduction in the numbers of neutrophils in drug-treated as compared to vehicle-treated animals. This data suggest that improved locomotor recovery in the drug treated group may be due to decreased white matter damage by neutrophils.

Blocking MMPs Improves Locomotor Recovery and Attenuates Histologically Assessible White Matter Damage.

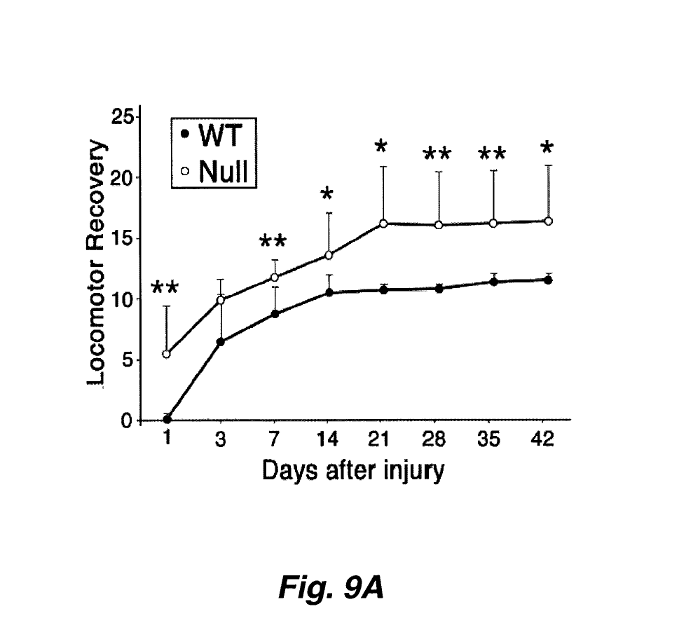

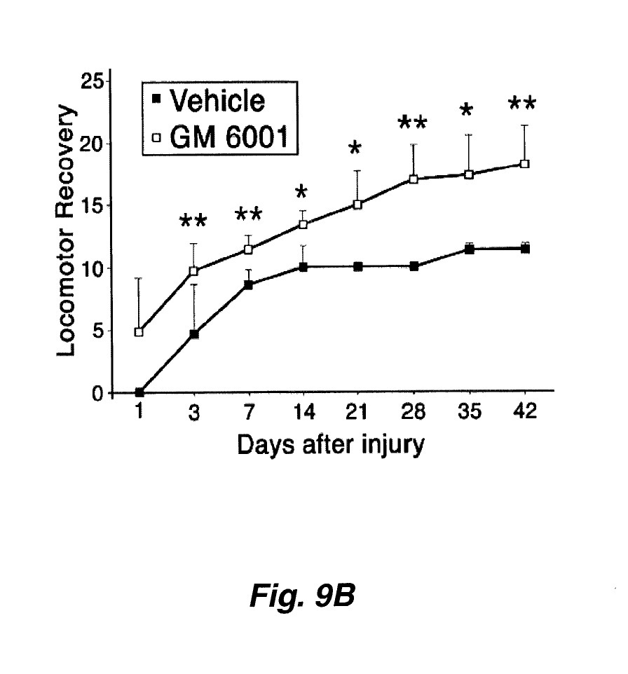

We next asked if MMP activity affected locomotor recovery after spinal cord injury using an open field testing paradigm, the BBB Locomotor Rating Scale, (Basso et al. 1995) J. Neurotrauma 12:1-21). There was significant locomotor recovery as early as 3 days post injury and weekly thereafter in MMP-9 null and in wild type mice that were treated with the MMP inhibitor GM 6001 as compared to their respective controls (FIG. 9).

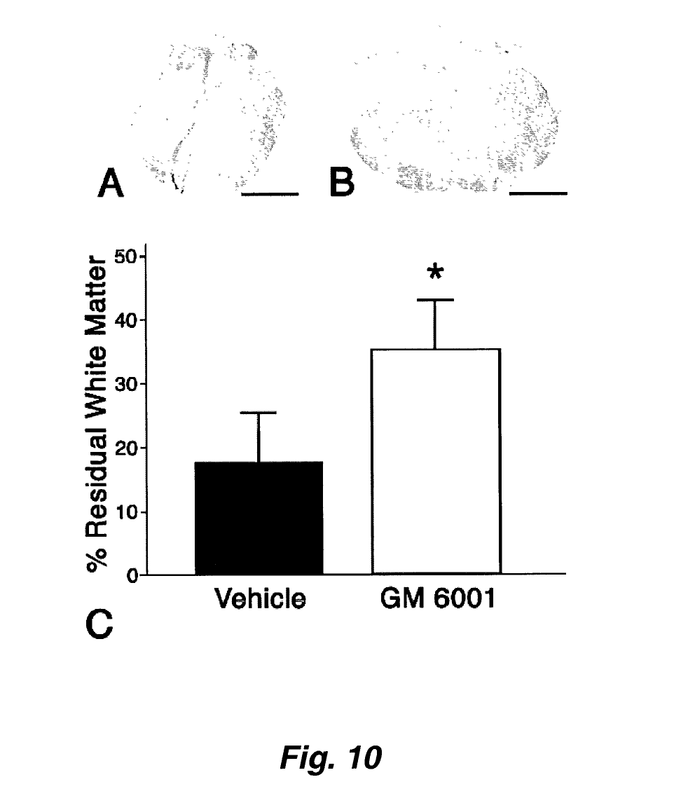

When the area of residual white matter was quantified in the injured spinal cords of wild type mice that were treated with the MMP inhibitor GM 6001 there was significant preservation of white matter as compared to the vehicle treated groups (FIG. 10). These data indicate that blocking MMP activity attenuates tissue damage and promotes locomotor recovery.

Discussion.

We report that spinal cord injured animals with a null mutation in MMP-9 exhibit reduced infiltration of neutrophils, stabilization of the blood-spinal cord barrier, and significant locomotor recovery as compared to wild type littermates. Moreover, similar observations were noted after pharmacologic inhibition of MMPs, beginning 3 hours post injury over a period of 3 days, a time frame coinciding with prominent disruption of the barrier and infiltration of neutrophils. Together, these exciting findings suggest that acute inhibition of MMPs may have efficacy as a therapeutic strategy for the treatment of human spinal cord injury.

Overview of MMP Functions

MMPs are important for extracellular matrix remodeling and are integral to morphogenesis, inflammation, cancer, and wound healing (Sternlicht (2001) Annu Rev Cell Dev Biol., 17:463-516; Yong et al. (2001) Nat Rev Neurosci 2:502-511). MMPs degrade components of the extracellular matrix, including fibrillar and non-fibrillar collagens, fibronectin, laminin, and glycoproteins and non-matrix substrates including serine proteinase inhibitors (Vu et al. (1998) Cell 93:411-422; Vu and Werb (1998) Gelatinase B: structure, regulation, and function. San Diego: Academic Press). MMP-9 is capable of degrading gelatin, collagens, elastin, vitronectin, and the major components of the basal lamina comprising the blood-brain barrier (Mun-Bryce and Rosenberg (1998a) J. Cerebral Blood Flow and Metabolism 18:1163-1172)..

MMP-9 in the Intact and Injured Spinal Cord.

MMP-9 expression was restricted to motoneurons and the meninges in the uninjured spinal cord. By 24 hours post injury, immunoreactive MMP-9 was expressed in vascular structures, astrocytes, neutrophils and microglia/macrophages. The change in expression of MMP-9 in the injured cord at 24 hours post injury corresponded to its prominent activation, as demonstrated with gelatin zymography. MMP-9 is regulated by several mechanisms: transcriptional control, secretion as an inactive zymogen subject to proteolytic activation, and inhibition by its endogenous inhibitor, TIMP-1 (Vu and Werb (1998) Gelatinase B: structure, regulation, and function. San Diego: Academic Press; Sternlicht (1999) P. 503-603 In: Extracellular Matrix Proteinases. In: Guidebook to the Extracellular Matrix, Anchor and Adhesion Proteins (Kreis T, Vale R, eds), pp Oxford University Press, N.Y.). Proteolysis by MMPs in normal or pathological states depends on the balance of proteinase to inhibitor (Sternlicht (1999) P. 503-603 In: Extracellular Matrix Proteinases. In: Guidebook to the Extracellular Matrix, Anchor and Adhesion Proteins (Kreis T, Vale R, eds), pp Oxford University Press, N.Y.). In the present study, TIMP-1 was unchanged during the period of maximal activity of MMP-9. This finding and the results from in situ zymography, which shows sites of net active protease, suggest active proteolysis by MMP-9 in the acutely injured spinal cord and establish the basis for defining the contribution of this protease to secondary damage.

MMPs and Inflammation After Spinal Cord Injury.

Neutrophils infiltrate the traumatized cord within the first several days post injury (Dusart Schwab (1994) Eur. J. Neurosci., 6:712-724; Taoka et al. (1997) Neuroscience 79:1177-1182; Carlson et al. (1998) Exp. Neurol., 151:77-88). We observed reduced numbers of neutrophils in the injured spinal cord at 24 hours post injury in MMP-depleted mice, a finding consistent with the role of MMP-9 in the transmigration of neutrophils from the vascular compartment (Pluznik et al. (1992) Exp. Hematol., 20:57-63; Delclaux et al. (1996) Am. J. Resp. Cell Mol. Biol., 14:288-295).

Our findings likewise implicate neutrophils in impaired locomotor recovery after spinal cord injury. We demonstrate decreased infiltration of neutrophils and improved locomotor recovery in animals treated over the first 3 days post injury, a period coinciding with maximal infiltration of neutrophils into the traumatized spinal cord. Although controversial (Bartholdi and Schwab (1995) Brain Res., 672:177-186), neutrophils have been implicated in secondary pathogenesis after spinal cord injury (Taoka et al. (1998) Brain Res., 799:264-269).

Neutrophils damage tissue by generating reactive oxygen species as well proteases, including MMPs (Weiss (1989) New Engl. J. Med., 320:365-376). MMP-9 is characterized by a broad substrate specificity that includes extracellular matrix proteins as well as nonmatrix proteins such as <1-proteinase inhibitor, the primary inhibitor of neutrophil elastase (Liu et al. (2000) Cell 102:647-655). MMP-9 promotes tissue damage either directly by disrupting structural proteins or indirectly by inactivating proteins such as <1-proteinase inhibitor (Banda et al. (1980) J. Exp. Med., 152:1563-1570; Sires et al. (1994) Biochem. Biophys. Res. Comm., 204:613-620; Liu et al. (2000) Cell 102:647-655). Its involvement with <1-proteinase inhibitor is of particular interest since neutrophil elastase degrades the perivascular extracellular matrix and its inhibition attenuates intraparenchymal hemorrhage and improves neurologic recovery after spinal cord injury (Armao et al. (1997) Brain Res. 767:259-264; Taoka et al. (1998) Brain Res., 799:264-269). Because <1-proteinase inhibitor-elastase complexes are chemotactic for neutrophils, inhibiting MMP-9 may also diminish further recruitment of neutrophils

The recruitment of neutrophils requires sequential appearance of several molecularly distinct chemoattractants (Chen et al. (2001) J. Clin. Invest., 108:1151-1158; Liu et al. (2000) Cell 102:647-655). We found that administration of an MMP inhibitor was highly effective in blocking neutrophil recruitment and tissue damage when administered hours after the injury. MMP-9 is also prominently expressed during macrophage infiltration after peripheral nerve injury. This suggests that MMPs in general, and MMP-9 in particular, play a significant role in the sustained phases of inflammatory cell recruitment.

MMPs and the Blood-spinal Cord Barrier

Increased activity of MMP-9 at 24 hours post injury coincided with prominent disruption of the blood-spinal cord barrier. Furthermore, this abnormal permeability was significantly reduced in MMP-depleted animals. MMP-9 has been implicated in blood-brain barrier disruption associated with inflammatory demyelinating disorders (Gijbels et al. (1994) J. Clin. Invest., 94:2177-2182; Rosenberg et al. (1994) Laboratory Investigation 71:417-422; Lim et al. (1996) J. Neurochemistry 67:251-259; Maeda and Sobel (1996) J. Neuropathology Exp. Neurology 55:300-309; Rosenberg et al. (1996) Neurology 46:1626-1632; Anthony et al. (1997) Neuropathology and Applied Neurobiology 23:406-415), hemorrhagic brain injury, intracerebral administration of cytokines and cerebral ischemia (Rosenberg et al. (1996a) J. Cerebral Blood Flow and Metabolism 16:360-366; Rosenberg and Navratil (1997) Neurology 48:921-926; Mun-Bryce and Rosenberg (1998) Am. J. Phpysiol., 274:R1203-1211; Rosenberg et al. (1998) Stroke 29:2189-2195; Fujimura et al. (1999) Brain Res., 842:92-100). Moreover, there is evidence that inhibition of MMPs blocks barrier disruption (Rosenberg and Navratil (1997) Neurology 48:921-926; Mun-Bryce and Rosenberg (1998b) Am. J. Phpysiol., 274:R1203-1211).

MMP-9 and other MMPs may influence the integrity of the blood-spinal cord barrier by degrading the basal lamina and/or tight junctions of endothelial cells. Leukocytes utilize MMPs during transmigration (Pluznik et al. (1992) Exp. Hematol., 20:57-63; Yong et al. (2001) Nat Rev Neurosci 2:502-511). MMPs degrade VE-cadherins and other cell-cell communication molecules (Sternlicht (2001) Annu Rev Cell Dev Biol., 17:463-516). The recruitment of leukocytes triggers signal transduction cascades leading to junctional disorganization and abnormal vascular permeability (Bolton et al. (1998) Neurosci., 86:1245-1257). The basal lamina surrounding endothelial cells plays a critical role in maintaining the integrity of the barrier in part by providing structural support to the endothelial cell (Kalaria (2000) Neurobiology of Aging 21:321-330; Farkas and Luiten (2001) Progr. Neurobiology 64:575-611). MMPs released from degranulating leukocytes may therefore compromise blood-brain barrier function and promote vasogenic edema.

MMPs and Recovery of Locomotor Function.

A critical finding of these studies is that MMP-9 null mice exhibited significant locomotor recovery compared to their wild type controls after spinal cord injury. This neuroprotection may be attributed to the early involvement of MMPs in secondary pathogenesis. This hypothesis is based upon our observation that animals, treated with an MMP inhibitor within the first 3 days post injury, likewise exhibited similar significant improvement in locomotor recovery.

Our studies suggest that the protection afforded by acute intervention with an MMP inhibitor targets early vascular responses associated with both disruption of the blood-spinal cord barrier and inflammation. Disruption of the barrier after spinal cord injury results in the influx of inflammatory cells and indiscriminate extravasation of molecules including plasma proteins (Noble and Wrathall (1989) Brain Res., 482:57-66; Popovich et al. (1996) Exp. Neurol., 142:258-275). This abnormal permeability exposes the spinal cord to the toxic effects of inflammatory cells, as well as to amino acids such as glutamate and glycine which, when present at high concentrations, can be toxic to cells (Schlosshauer (1993) Bioessays 15:341-346).

We demonstrate significant neuroprotection of white matter in animals treated with the MMP inhibitor as compared to the vehicle controls. Such protection may in part account for the improved locomotor recovery. Similar findings of neuroprotection have been reported after cerebral infarction, produced by systemic blockade of MMP-9 with a neutralizing antibody (Romanic et al. (1998) Stroke 29:1020-1030). In this study, the administration of the MMP inhibitor was initiated at 3 hours post injury and was maintained for 3 days post injury. The delay in treatment for 3 hours was selected for its potential relevance to the spinal cord injured patient. Moreover, the limited duration of treatment, from 3 hours to 3 days, more precisely defined the contribution of MMPs to early pathogenesis and the extent to which this acute inhibition would influence white matter pathology and locomotor recovery. We found that the MMP inhibitor not only stabilized the barrier but also reduced the infiltration of neutrophils. Thus, neuroprotection and the resulting improvement in locomotion may be due to decreased acute secondary damage.

It also possible that MMP-9, produced by macrophages, damages myelinated axons that were originally spared by the initial injury. Macrophages are integral to delayed demyelination of those populations of axons that have withstood the traumatic insult (Blight (1985) Central Nervous System Trauma 2:299-315; Blight (1994) Neurosci., 60:263-273). Macrophages are in close proximity to the myelin sheaths of axons and infiltrate the myelin lamellae of normal appearing axons (Gledhill et al. (1973) Exp. Neurol., 38:472-487; Griffiths and McCulloch (1983) J. Neurol. Sci., 58:335-349; Blight (1985) Central Nervous System Trauma 2:299-315). This relationship is significant since both macrophages and microglia secrete MMP-9 and other MMPs.

There is evidence that MMP-9 contributes to demyelination. There is widespread expression of MMP-9 in macrophages and reactive astrocytes in certain demyelinating diseases (Cuzner et al. (1996) J. Neuropathology Exp. Neurol., 55:1194-1204; Maeda and Sobel (1996) J. Neuropathology Exp. Neurology 55:300-309). Moreover, MMP expression is reduced in those regions exhibiting inactive lesions (Maeda and Sobel (1996) J. Neuropathology Exp. Neurology 55:300-309). This suggests that MMP expression correlates closely with localization of active demyelination. MMP-9 cleaves myelin basic protein (Gijbels et al. (1993) J. Neurosci. Res., 36:432-440) and thus may contribute to the disintegration of the myelin sheath. Future studies will be required to more specifically identify the role of contribution of MMP-9, produced by macrophages and glia, in axonal degeneration after spinal cord injury. In the shorter term, our studies suggest that MMP inhibitors administered in the first few hours after spinal cord injury could attenuate the poor outcomes due to the secondary damage.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of reducing neurological damage following trauma to nervous tissue in a mammal, said method comprising inhibiting activity or expression of a matrix metalloproteinase in said mammal before, during, or after said trauma.

2. The method of claim 1, wherein said method comprises administering to said mammal a matrix metalloproteinase inhibitor (MMPI) during or after said trauma.

3. The method of claim 2, wherein said matrix metalloproteinase inhibitor (MMPI) is selected from the group consisting of a 4-biarylbutyric acid derivative, a 5-biarylpentanoic acid derivative, Fenbufen, peptide MMPIs, a hydroxamic acid, a tricyclic butyric acid derivative, a biphenyl butyric acid derivative, a heterocyclic substituted phenyl butyric acid derivative, a sulfonamide derivative, a succinamide MMP inhibitor, a sulfonated amino acid derivatives, and a neutralizing anti-MMP antibody.

4. The method of claim 2, wherein said matrix metalloproteinase inhibitor (MMPI) is selected from the group consisting of BAY12-9566, batimastat (BB-94), TIMP-1, prinomastat (AG-3340), and RO 31-9790.

5. The method of claim 2, wherein said MMPI is provided in a unit dosage form at a concentration sufficient to inhibit neurological damage following trauma to nervous tissue in said mammal.

6. The method of claim 2, wherein said trauma is selected from the group consisting of ischemia, spinal cord injury, cranial injury, physical trauma to the head or spinal cord; a brain concussion; ischemic stroke caused by thrombosis or embolism; cerebral hemorrhage, general circulatory failure, circulatory disruption caused by cardiac arrest, hemodynamic shock caused by loss of blood due to injury or hemorrhage elsewhere in the body; vasculatory damage caused by vascular disease, bacterial infection, viral infection, fungal infection, cerebral or spinal tumors, glial cell swelling, hypoxic injury to the brain caused by respiratory disruption, and post-operative brain injury or stress.

7. The method of claim 2, wherein said trauma is spinal cord injury.

8. The method of claim 2, wherein said MMPI is an inhibitor of MMP-9.

9. The method of claim 2, wherein said mammal is a human.

10. The method of claim 2, wherein said mammal is a non-human mammal.

11. The method of claim 2, wherein said mammal is a human afflicted with or following a stroke.

12. The method of claim 2, wherein said mammal is a human afflicted with a spinal cord injury.

13. The method of claim 2, wherein said administering is for up to 5 days following said trauma.

14. The method of claim 1, wherein said method comprises administering to said mammal an agent that inhibits expression of a matrix metalloproteinase, with the proviso that said agent is not a glucocorticoid.

15. The method of claim 14, wherein the agent is not methylprednisolone.

16. The method of claim 8, wherein said MMPI is not an inhibitor of MMP-2 activity.

17. The method of claim 16, wherein said MMPI is not an inhibitor of the activity of any matrix metalloproteinase other than MMP-9.

18. The method of claim 16, wherein said trauma is spinal cord injury.

19. The method of claim 16, wherein said trauma is brain injury.

20. The method of claim 16, wherein said trauma is motor nerve injury.

21. The method of claim 16, wherein said trauma is sensory nerve injury.

22. The method of claim 1 or 14, wherein said method comprises administering to said mammal a MMP-9 inhibitor and a prophylactically or therapeutically effective amount of one or more anti-inflammatory agents during or after said trauma.

23. The method of claim 22, wherein at least one anti-inflammatory agent is a non-steroidal anti-inflammatory drug.

24. The method of claim 23, wherein the non-steriodal anti-inflammatory drug is aspirin, ibuprofen, diclofenac, nabumetone, naproxen, or ketoproten.

25. The method of claim 1 or 24, wherein said method comprises administering to said mammal a matrix metalloproteinase inhibitor (MMPI) and a prophylactically or therapeutically effective amount of one or more anti-convulsive agents during or after said trauma.

26. The method of claim 25, wherein the anti-convulsive agent is carbamazepine (Tegretol®), phenobarbital, primidone, phenytoin (Dilantin®), valproic acid (Depakote®), ethosuximide, clonazepam, levitracetam, gabapentin, gabatril, lamotrigine, oxcarbazepine or topiramate.

27. A method of reducing abnormal vascular permeability associated with spinal cord injury, said method comprising administering to a mammal in need thereof a matrix metalloproteinase inhibitor (MMPI) in an amount sufficient to reduce abnormal vascular permeability associate with or following said spinal cord injury.

28. The method of claim 27, wherein said matrix metalloproteinase inhibitor (MMPI) is selected from the group consisting of a 4-biarylbutyric acid derivative, a 5-biarylpentanoic acid derivative, Fenbufen, peptide MMPIs, a hydroxamic acid, a tricyclic butyric acid derivative, a biphenyl butyric acid derivative, a heterocyclic substituted phenyl butyric acid derivative, a sulfonamide derivative, a succinarmide MMP inhibitor, a sulfonated amino acid derivatives, and a neutralizing anti-MMP antibody.

29. The method of claim 27, wherein said matrix metalloproteinase inhibitor (MMPI) is selected from the group consisting of BAY12-9566, batimastat (BB-94), TIMP-1, prinomastat (AG-3340), and RO 31-9790.

30. The method of claim 27, wherein said MMPI is provided in a unit dosage form at a concentration sufficient to inhibit abnormal vascular permeability following spinal cord injury.

31. The method of claim 27, wherein said MMPI is an inhibitor of MMP-9.

32. The method of claim 27, wherein said mammal is a human.

33. The method of claim 27, wherein said mammal is a non-human mammal.

34. The method of claim 27, wherein said mammal is a human afflicted with or following a stroke.

35. The method of claim 27, wherein said mammal is a human afflicted with a spinal cord injury.

36. The method of claim 31, wherein said MMPI is not an inhibitor of MMP-2 activity.

37. The method of claim 16, wherein said MMPI is not an inhibitor of the activity of any matrix metalloproteinase other than MMP-9.

38. The method of claim 16, wherein said spinal cord injury comprises motor nerve injury.

39. The method of claim 16, wherein said spinal cord injury comprises sensory nerve injury.

40. The method of claim 27 or 14, wherein said method comprises administering to said mammal a MMP-9 inhibitor and a prophylactically or therapeutically effective amount of one or more anti-inflammatory agents during or after said trauma.

41. The method of claim 22, wherein at least one anti-inflammatory agent is a non-steroidal anti-inflammatory drug.

42. The method of claim 23, wherein the non-steriodal anti-inflammatory drug is aspirin, ibuprofen, diclofenac, nabumetone, naproxen, or ketoproten.

43. The method of claim 27 or 24, wherein said method comprises administering to said mammal a matrix metalloproteinase inhibitor (MMPI) and a prophylactically or therapeutically effective amount of one or more anti-convulsive agents during or after said trauma.

44. The method of claim 25, wherein the anti-convulsive agent is carbamazepine (Tegretol®), phenobarbital, primidone, phenytoin (Dilantin®), valproic acid (Depakote®), ethosuximide, clonazepam, levitracetam, gabapentin, gabatril, lamotrigine, oxcarbazepine or topiramate.

45. A method of improving recovery of neurological function following injury to neurological tissue, said method comprising administering to a mammal in need thereof a matrix metalloproteinase inhibitor (MMPI) in an amount sufficient to improve recovery of neurological function following said injury.

46. The method of claim 45, wherein said injury is spinal cord injury.

47. The method of claim 46, wherein said recovery comprises recovery of locomotor function.

48. The method of claim 45, wherein said matrix metalloproteinase inhibitor (MMPI) is selected from the group consisting of a 4-biarylbutyric acid derivative, a 5-biarylpentanoic acid derivative, Fenbufen, peptide MMPIs, a hydroxamic acid, a tricyclic butyric acid derivative, a biphenyl butyric acid derivative, a heterocyclic substituted phenyl butyric acid derivative, a sulfonamide derivative, a succinamide MMP inhibitor, a sulfonated amino acid derivatives, and a neutralizing anti-MMP antibody.

49. The method of claim 45, wherein said matrix metalloproteinase inhibitor (MMPI) is selected from the group consisting of BAY12-9566, batimastat (BB-94), TIMP-1, prinomastat (AG-3340), and RO 31-9790.

50. The method of claim 49, wherein said MMPI is provided in a unit dosage form at a concentration sufficient to promote recovery of locomotor function following spinal cord injury.

51. The method of claim 45, wherein said injury can comprise an injury selected from the group consisting of ischemia, spinal cord injury, cranial injury, physical trauma to the head or spinal cord; a brain concussion; ischemic stroke caused by thrombosis or embolism; cerebral hemorrhage, general circulatory failure, circulatory disruption caused by cardiac arrest, hemodynamic shock caused by loss of blood due to injury or hemorrhage elsewhere in the body; vasculatory damage caused by vascular disease, bacterial infection, viral infection, fungal infection, cerebral or spinal tumors, glial cell swelling, hypoxic injury to the brain caused by respiratory disruption, and post-operative brain injury or stress.

52. The method of claim 45, wherein said MMPI is an inhibitor of MMP-9.

53. The method of claim 45, wherein said mammal is a human.

54. The method of claim 45, wherein said mammal is a non-human mammal.

55. The method of claim 45, wherein said mammal is a human afflicted with or following a stroke.

56. The method of claim 45, wherein said mammal is a human afflicted with a spinal cord injury.

57. The method of claim 52, wherein said MMPI is not an inhibitor of MMP-2 activity.

58. The method of claim 16, wherein said MMPI is not an inhibitor of the activity of any matrix metalloproteinase other than MMP-9.

59. The method of claim 16, wherein said trauma is spinal cord injury.

60. The method of claim 16, wherein said trauma is brain injury.

61. The method of claim 16, wherein said trauma is motor nerve injury.

62. The method of claim 16, wherein said trauma is sensory nerve injury.

63. The method of claim 45 or 14, wherein said method comprises administering to said mammal a MMP-9 inhibitor and a prophylactically or therapeutically effective amount of one or more anti-inflammatory agents during or after said trauma.

64. The method of claim 22, wherein at least one anti-inflammatory agent is a non-steroidal anti-inflammatory drug.

65. The method of claim 23, wherein the non-steriodal anti-inflammatory drug is aspirin, ibuprofen, diclofenac, nabumetone, naproxen, or ketoproten.

66. The method of claim 45 or 24, wherein said method comprises administering to said mammal a matrix metalloproteinase inhibitor (MMPI) and a prophylactically or therapeutically effective amount of one or more anti-convulsive agents during or after said trauma.

67. The method of claim 25, wherein the anti-convulsive agent is carbamazepine (Tegretol®), phenobarbital, primidone, phenytoin (Dilantin®), valproic acid (Depakote®), ethosuximide, clonazepam, levitracetam, gabapentin, gabatril, lamotrigine, oxcarbazepine or topiramate.

68. A kit for reducing neurological damage following trauma to nervous tissue in a mammal, said kit comprising:
a matrix metalloproteinase inhibitor (MMPI): and
instructional materials teaching the use of said matrix metalloproteinase inhibitor for reducing neurological damage following trauma to nervous tissue in a mammal.

69. The kit of claim 68, wherein said matrix metalloproteinase inhibitor (MMPI) is selected from the group consisting of a 4-biarylbutyric acid derivative, a 5-biarylpentanoic acid derivative, Fenbufen, peptide MMPIs, a hydroxamic acid, a tricyclic butyric acid derivative, a biphenyl butyric acid derivative, a heterocyclic substituted phenyl butyric acid derivative, a sulfonamide derivative, a succinamide MMP inhibitor, a sulfonated amino acid derivatives, and a neutralizing anti-MMP antibody.

70. The kit of claim 68, wherein said matrix metalloproteinase inhibitor (MMPI) is selected from the group consisting of BAY12-9566, batimastat (BB-94), TIMP-1, prinomastat (AG-3340), and RO 31-9790.

71. The kit of claim 68, wherein said MMPI is provided in a unit dosage form at a concentration sufficient to inhibit secondary neurological damage following said trauma.

72. The kit of claim 68, wherein said trauma is selected from the group consisting of ischemia, spinal cord injury, cranial injury, physical trauma to the head or spinal cord; a brain concussion; ischemic stroke caused by thrombosis or embolism; cerebral hemorrhage, general circulatory failure, circulatory disruption caused by cardiac arrest, hemodynarmic shock caused by loss of blood due to injury or hemorrhage elsewhere in the body; vasculatory damage caused by vascular disease, bacterial infection, viral infection, fungal infection, cerebral or spinal tumors, glial cell swelling, hypoxic injury to the brain caused by respiratory disruption, and post-operative brain injury or stress.

73. The kit of claim 68, wherein said trauma is spinal cord injury.

74. The kit of claim 68, wherein said MMPI is an inhibitor of MMP-9.

75. The kit of claim 68, wherein said mammal is a human.

76. The kit of claim 68, wherein said mammal is a non-human mammal.

77. The kit of claim 68, wherein said mammal is a human afflicted with or following a stroke.

78. The kit of claim 68, wherein said mammal is a human afflicted with a spinal cord injury.

79. In a mammal diagnosed as having or as at risk from secondary neurological damage, an exogenously applied inhibitor of a matrix metalloproteinase activity or expression.

80. The mammal of claim 79, wherein said mammal is not diagnosed as having a cancer.

81. The mammal of claim 79, wherein said inhibitor is an inhibitor of MMP-9 expression or activity.
